(19) United States
(12) Patent Application Publication
(10) Pub. No.: US 2023/0390312 A1
(43) Pub. Date: Dec. 7, 2023

(54) NOVEL THERAPEUTIC COMBINATIONS COMPRISING DERIVATIVES OF OXAZAPHOSPHORINES FOR THE TREATMENT OF CANCER

(71) Applicant: INSTITUT GUSTAVE ROUSSY, VILLEJUIF (FR)

(72) Inventors: ANGELO PACI, MEUDON (FR); NATHALIE CHAPUT, PARIS (FR); JEAN-LUC PERFETTINI, MEAUX (FR); JULIA DELAHOUSSE, CARRIERES SOUS POISSY (FR)

(21) Appl. No.: 17/767,474

(22) PCT Filed: Oct. 9, 2020
(86) PCT No.: PCT/EP2020/078458

§ 371 Date: Apr. 8, 2022

(30) Foreign Application Priority Data
Oct. 11, 2019 (EP) 19306332.8

(51) Int. Cl. A61K 31/675; A61K 45/06; A61P 35/00; A61P 37/02

(52) U.S. Cl. CPC A61K 31/675; A61K 45/06; A61P 35/00; A61P 37/02; A61K 2039/505

(57) ABSTRACT

The present invention relates to novel therapeutic combinations comprising an oxazaphosphorine derivative and an immune checkpoint modulator for the treatment or the prevention of cancers.

The present invention relates to novel therapeutic combinations useful for the treatment of cancers.

### BACKGROUND OF THE INVENTION

Oxazaphosphorines belong to alkylating agents that have been widely used in routine clinical practices to treat several types of cancer from soft tissue tumor to lymphoma. They are still the corner stone of several polychemotherapy protocols. Oxazaphosphorines include ifosfamide (IFO), cyclophosphamide (CPA) and trofosfamide, which have an isomeric structure containing one, two or three chloroethyl groups bound to the nitrogen atoms. As prodrugs, these compounds require a metabolic activation fulfilled by specific liver cytochrome P450 (CYP). This activation produces hydroxylated intermediates which by a ring opening mechanism liberate the active drug, namely the nitrogen mustard which displays cytotoxicity by DNA crosslinks. The main activation pathway of IFO is carried out by CYP3A4 and involves an oxidation reaction on the C-4 carbon atom, leading to 4-hydroxy-ifosfamide (4-HO-IFO). 4-HO-IFO gives rise to the alkylating mustard concomitantly with acrolein through tautomeric equilibrium and retro-Michael process. Acrolein is responsible of the urological toxicity characterized by hemorrhagic cystitis. In addition, oxazaphosphorines may also cause neurotoxicity and nephrotoxicity due to the release of chloroacetaldehyde, a metabolite produced by oxidation of the side chains of the molecules via the action of cytochrome, in particular CYP2B6. It is assessed that only 10 to 50% of IFO administrated to the patient is transformed in the desired alkylating mustard while 50% to 90% of the administered IFO release nephrotoxic and neurotoxic chloroacetaldehyde (CAA) (Goren, Lancet, 1986, 2(8517):1219-20; Ben Abid, Oncologie, 2007, 9(11):751-7). It was observed that the toxicities of oxazaphosphorines increase in high-dose setting protocols. For instance, Le Cesne et al. showed that IFO at high dose administration (cumulative dose of 12 000 mg/m2) was effective in patients with advanced refractory soft tissue but resulted in major toxicities (Le Cesne, JClinOncol, 1995, 13(7):1600-8). It is also the case in pediatric patients. A clinical study performed in children with osteosarcoma, previously treated with conventional chemotherapy, showed that the administration of IFO at high dose (cumulative dose of 14 000 mg/m²) resulted in an improved disease-free survival in 30% of patients but with severe nephrotoxicity in a quarter of patients (Berrak, Pediatr Blood Cancer, 2005, 44(3):215-9). Consequently, the increase of the therapeutic index of oxazaphosphorines is a an important clinical issue.

Several research teams have been seeking methods to circumvent the toxicity oxazophosphorines

The co-administration of sodium mercaptoethanesulfonate is proposed in order to attenuate acrolein-based toxicity. On the other hand, pharmacomodulation of oxazaphosphorines has been also investigated to circumvent these toxicities. Chemical oxidation of the C-4 carbon center has been proposed in order to provide pre-activated analogues able to release the alkylating mustards without undergoing metabolization by cytochrome P450. Many derivatives have been already prepared such as 4-methoxy derivatives (Paci et al., 2001, Bioorg Med Chem Lett, 11, 1347-1349) but most of them were found either too unstable for further development or with no advantage over the use of IFO.

Patent application WO2012/076824 discloses several ifosfamide derivatives including SQ-IFO and SQ-thio-IFO which comprise a squalenoyl radical at C-4 carbon. These compounds were shown to display cytotoxicity on several cancerous cells and to be able to self-organize into nanoparticles thanks to its long hydrophobic tail. Patent application WO2015/173367 discloses derivatives of oxazaphosphorines comprising a geranyl radical at C-4 carbon, such as geranyloxy-Ifosfamide (geranyloxy-IFO, G-IFO). This compound was shown to be cytotoxic on a large panel of tumor cells in vitro, and to prevent tumor growth in a murine model of rhabdomyosarcoma. It was also shown that, when injected in mice by intravenous route, geranyloxy-IFO was rapidly converted into 4-hydroxy-ifosfamide metabolite which spontaneously releases the alkylating mustard.

However, there is still a need of new therapeutic methods for the treatment of cancers.

### SUMMARY OF THE INVENTION

The invention relates to the use of an oxazaphosphorine derivative of formula (I):

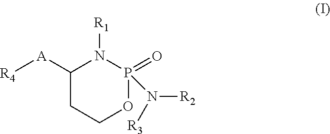

wherein:
A is O, O—O, S, NH, NR₅ with R₅ is an alkyl group, preferably a C₁-C₃ alkyl group, or a linker group having a molecular weight up to 500 g·mol⁻¹, more preferably lower than 400 g·mol⁻¹,
R₁, R₂ and R₃ are independently selected from the group consisting of —H, —CH(CH₃)—CH₂—X and —(CH₂)₂—X, wherein X is an halogen atom, preferably Cl, Br or I, and more preferably Br or Cl,
R₄ is H or a saturated or unsaturated chain of 2 to 30 carbon atoms optionally interrupted by one or several heteroatoms such as S, O and NH, and optionally substituted by one or several substituents independently selected from the group consisting of halogen (e.g. F, Cl, Br, I), CN, CF₃, OH, C₁-C₆ alkyl, C₁-C₆ hydroxyalkyl, C₁-C₆ alkyloxy, C₁-C₆ aminoalkyl, C₁-C₆ halogenoalkyl, —C₂-C₆ alkoxy alkyl, —C(O)OR, —OC(O)R, —OC(O)OR, —C(O)R, —NHC(O)—NH—R, —NH—C(O)—R, —C(O)—NH—R, —NRR′, —C(O)NRR′, —NC(O)R, —NRC(O)R′, and —SR, wherein R and R′ are independently selected from H and C₁-C₆ alkyl,
   and pharmaceutically acceptable salt or solvate thereof,
   in combination with an immune checkpoint modulator to treat or prevent a cancer.

In some embodiments, the oxazaphosphorine derivative is of formula (Ia):

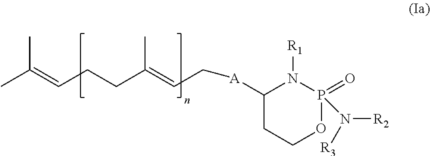

wherein
n is an integer from 0 to 3, preferably 1 or 2,
A, R₁, R₂ and R₃ are as defined for the compound of formula (I) in claim 1
   and pharmaceutically acceptable salt and solvate thereof.

In some additional embodiments, the oxazaphosphorine derivative is a compound of formula (Ia), wherein
n is 1 or 2,
A is selected from the group of O, O—O, S, and —NH—, or comprises, or consists of, a spacer moiety selected from the group consisting of:
   natural or non-natural amino acids, dipeptides, and derivatives thereof;
   polyether groups, such as polyethylene glycol or polypropylene glycol, preferably comprising from 2 to 6 monomers e.g. 2, 3, or 4 monomers;
   hydrazone linkers, e.g. of formula —CR₇═N—NH—C(O)— wherein R₇ is H or a C₁-C₆, preferably C₁-C₃ alkyl,
—O—(C═S)—S—, —ONR₇—, —NR₇O—, with R₇ being H or a C₁-C₆ preferably C₁-C₃ alkyl,
Y₁—(CH₂)ₙ—Y_{2,} with n is an integer from 1 to 8, wherein Y₁ and Y₂ are independently selected from —O—, —S—, —OC(O)—, —C(O)O—, —OC(O)—O—, —C(O)NR₇—, NR₇C(O)—, —OC(S)S—, —SC(S)O— —NR₇—, —ONR₇—, —NR₇O—, NR₇C(S)S—, —SC(S)NR₇—
and

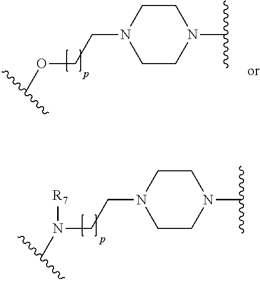

wherein R₇ is selected from the group of H and C₁-C₆, preferably C₁-C₃ alkyl, and p is an integer from 0 to 8, preferably 1, 2 or 3, and
R₁, R₂ and R₃ are such that one of R₁, R₂ and R₃ is H and the two other remaining groups are independently selected from —CH(CH₃)—CH₂—X and —(CH₂)₂—X, with X preferably being Cl or Br.

In some embodiments, the oxazaphosphorine of formula (I) or (Ia) is such that A is O, O—O, S or NH, or is a moiety selected from the group consisting of:
—O—(C═S)—S—, —ONR₇—, —NR₇O—, with R₇ is H or a C₁-C₃ alkyl, preferably CH₃
Citrulline, lysine, ornithine, alanine, phenylalanine, cysteine, glycine, valine, leucine and dipeptides thereof such as valine-citrulline,
Y₁—(CH₂)ₙ—Y₂, and
Y₁—(CH₂—CH₂—O)ₐ—CH₂—CH₂—Y₂
   wherein Y₁ and Y₂ are as defined above, preferably independently selected from O, NR₇, S, OC(O), C(O)O, NHCO, CONH with R₇ is H or a C₁-C₃ alkyl, preferably —CH₃, n is an integer from 1 to 8, preferably 1, 2, 3, or 4 and a is an integer from 1 to 3.

In some other embodiments, the oxazaphosphorine of formula (I) or (Ia) is such that R₁, R₂ and R₃ are independently selected from the group consisting of —H, and —CH(CH₃)—CH₂—X, wherein X is an halogen atom, preferably Cl, Br or I, and more preferably Br or Cl.

In other embodiments, the oxazaphosphorine of formula (I) or (Ia) is such that R₁, R₂ and R₃ are independently selected from the group consisting of —H, and —CH₂—CH₂—X, wherein X is an halogen atom, preferably Cl, Br or I, and more preferably Br or Cl.

In some embodiments, the oxazaphosphorine derivative may be selected from the group of compounds of formula (IIa) and of formula (IIb):

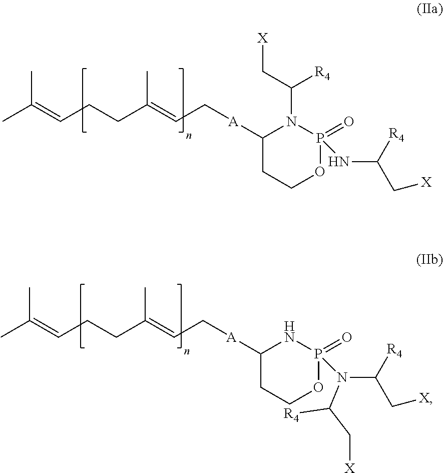

wherein
n is 1 or 2,
R₄ is H or CH₃,
X is Cl or Br, and
A is selected from the group consisting of O, S, —NH—, cysteamine linker, valine-citrulline linker and cysteine linker,
   and pharmaceutically acceptable salts and solvates thereof.

For instance, the oxazaphosphorine derivative is selected from the group consisting of:

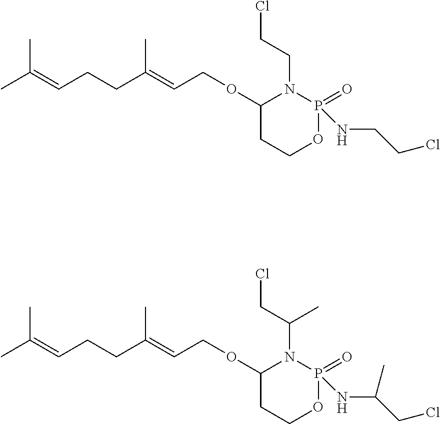 [SMILES: CC(C)=CCC/C(C)=C/COC1CCOP(=O)(NCCCl)N1CCCl ; CC(C)=CCC/C(C)=C/COC1CCOP(=O)(NC(C)CCl)N1C(C)CCl]

and pharmaceutically acceptable salts and solvates thereof.

The immune checkpoint modulator may be an immune checkpoint modulator of an inhibitory immune checkpoint pathway. For instance the immune checkpoint modulator can be an inhibitor of an immune checkpoint pathway selected from CTLA-4, PD-1, LAG-3, TIM-3, TIGIT and 2B4/CD244 immune checkpoint pathways, preferably an inhibitor of CTLA4 immune checkpoint pathway and PD1 immune checkpoint pathway.

For instance, the immune checkpoint modulator may be selected from the group consisting of an anti-PD1 antibody, an anti-PD-L1 antibody, an anti-CTLA4 antibody, anti-TIGIT and combinations thereof. Example of such immune checkpoint modulators encompass, without being limited to pembrolizumab (Keytruda®), nivolumab (Opdivo®), cemiplimab (Liptayo®), camrelizumab, sintilimab, spartalizumab, Tislelizumab, pidilizumab, JS001, avelumab (Bavencio®), atezolizumab (Tecentriq®), durvalumab (Imfinzi®), BMS936559, MDX-1105, KN305, ipilimumab (Yervoy®), tremelimumab, tiragulomab, vibostolimab, variants thereof, antigen-binding fragments thereof and combinations thereof.

In another embodiment, the immune checkpoint modulator is an OX40 agonist.

In a further embodiment, the immune checkpoint modulator is selected from LAG3 inhibitors and TIM-3 inhibitors, e.g. anti-LAG3 antibodies and anti-TIM-3 antibodies.

In some embodiments of the invention, the oxazaphosphosphorine derivative is geranyloxy-IFO and the immune checkpoint modulator is selected from PD1 inhibitors and PD-L1 inhibitors. For instance, the immune checkpoint modulator may be selected from the group consisting of pembrolizumab, nivolumab, variants thereof, antigen-binding fragments thereof and combinations thereof.

The oxazaphosphorine derivative and the immune checkpoint modulator may be administered to the subject simultaneously, successively or separately to the subject by the same administration route or by different administration routes.

The cancer may be of any type and can be selected from the group consisting of the chronic leukemias, acute lymphocytic leukemias, Hodgkin's disease, Hodgkin's and non-Hodgkin lymphomas, cancers of the lung, breast cancer including triple negative breast cancer, genitourinary cancers such as cancers of prostate, bladder, testis, uterine cervix or ovaries, sarcomas such as osteosarcoma and soft tissue sarcoma including pediatric soft tissue sarcoma, neuroblastomas, myelomas, Merkel-cell carcinoma and melanomas.

The invention also relates to a pharmaceutical composition for use in the treatment or the prevention of cancer, which comprises an oxazaphosphorine derivative as defined above, and an immune checkpoint modulator preferably as defined above.

A further object of the invention is a pharmaceutical kit for use in the treatment or the prevention of cancer, which comprises a first component comprising an oxazaphosphorine derivative, preferably as defined above, and a second component comprising an immune checkpoint modulator as defined above.

### FIGURES

FIG. 1 shows the metabolism of IFO and G-IFO in vivo.

FIG. 2. Low doses of G-IFO (geranyloxy-IFO) promoted T cell immunity and delayed tumor-growth in mice. MCA205 tumor-bearing mice were treated with a single i.p. injection of G-IFO (eq. 100 mg/kg) or CPA (100 mg/kg) or vehicle (DMSO/Tween 80/NaCl 0.9% (5/5/90, v/v/v)). (A) Seven days later, mice are sacrificed and spleens were collected. Lymphocytes were detected in the spleen after mechanic dissociation using flow cytometry methods. Absolute number of splenocytes, T cells, CD8+ T cells, CD4+ T cells and Treg cells. Graphs depict data from one experiment (n=3-4 mice/group). Median values with interquartile are presented. (B) Seven days later, mice are sacrificed and tumors were collected. Lymphocytes were detected in the tumor after mechanic dissociation using flow cytometry methods. Absolute number of splenocytes, T cells, CD8+ T cells, CD4+ T cells, Treg cells and ratio CD8+ T cells/Treg. Graphs depict data from one experiment (n=6 mice/group). Median values with interquartile are presented. (C) Seven days after treatment, mice were sacrificed and spleens were collected. Splenocytes were incubated with anti-CD3ε for 48 h at 37° C. Supernatants were harvested and concentrations of (left panel) IFNγ, (middle panel) IL-17A and (right panel) IL-6 were analyzed by ELISA. Graphs depict data from one experiment (n=6 mice/group). Medians with interquartile are shown. (D) Tumor volume was measured every 2-3 days, VTDi corresponds to the tumor volume the day of treatment initiation and VTDx correspond to the tumor volume. VTDx to VTDi ratio (VTDx/VTDi) is depicted from one experiment (n=6 mice/group). Graph depicts meant SEM. (A,B,C) Statistical analysis using Kruskal-Wallis test indicated significant differences at 95% CI. (D). Statistical analysis using 2 ways ANOVA test indicated significant differences at 95% CI. (A,B,C,D) No adjustment for multiple comparisons was made because of the exploratory component of the analyses. *, p<0.05; **, p<0.01; ***, p<0.001; ****, p<0.0001.

FIG. 3. The combination therapy anti-PD1 mAbs and G-IFO induced potent antitumor effect. MCA205 tumor-bearing mice were injected with a single i.p. injection of IFO 150 at low dose (150 mg/kg) or at high dose (300 mg/kg), G-IFO at low dose (eq. 100 mg/kg) or vehicle. Combination with anti-PD1 Mabs or its isotype control IgG2 has been performed with three i.p. injection at 200 or 250 μg/mouse. Grey arrow represents vehicle or chemotherapy injection; Black arrow represents IgG2 or Anti-PD1 injection. Tumor volume was measured every 2-3 days; Tumor volume was measured every 2-3 days. VTDx correspond to the tumor volume at the day X. Mice were sacrificed when they reached boundary points, as described in Methods. (A) Graphs depicted VTDx to VTDi ratio (VTDx/VTDi) as mean±SEM (n=6 mice per group) for groups treated in combination with isotype control IgG2 or anti-PD1 mAbs. (upper panel) Kinetic tumor growth and (lower panel) VTD23/VTDi are depicted. Statistical analysis using two-way ANOVA test indicated significant differences at 95% CI. No adjustment for multiple comparisons was made because of the exploratory component of the analyses. *, p<0.05; **, p<0.01; ***, p<0.001; ****, p<0.0001. (B) Graph depicted time to reach 5 fold the initial volume. Median values with interquartile are presented. Statistical analysis using Mann-Whitney test indicated significant differences at 95% CI. *, p<0.05; **, p<0.01; ***, p<0.001; ****, p<0.0001.

FIG. 4. Dose-dependent B cell depletion in the spleen of tumor-bearing mice treated with IFO, CPA or G-IFO. C₅₇Bl/6 were injected with a single i.p. injection of IFO (150 mg/kg) or CPA (CPM 100 mg/kg) or G-IFO (eg. 100 or 150 mg/kg) or vehicle (DMSO/Tween 80/NaCl 0.9% (5/5/90, v/v/v). Seven days later, mice are sacrificed, and spleens were collected. B cells were detected and quantified in the spleen after mechanic dissociation using flow cytometry methods.

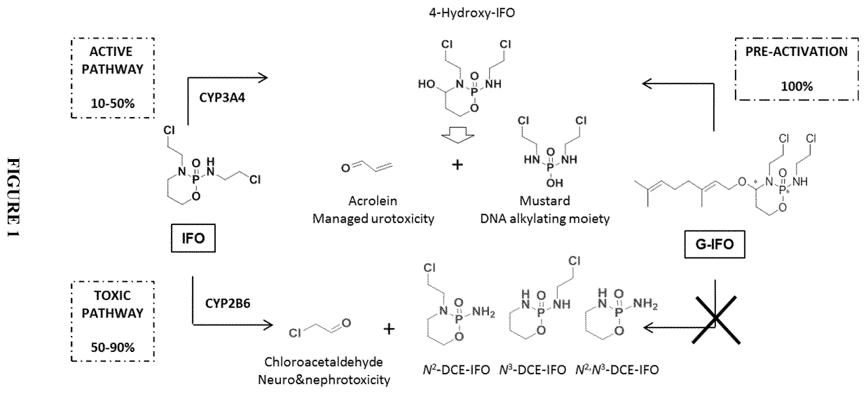

### DETAILED DESCRIPTION OF THE INVENTION

The present invention relates to a new combination therapy with an oxazaphosphorine derivative and an immune checkpoint modulator for treating or preventing a cancer.

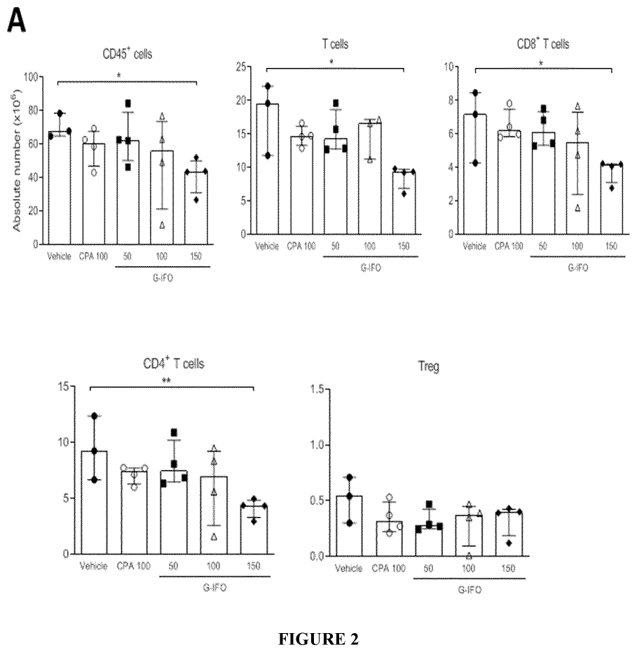

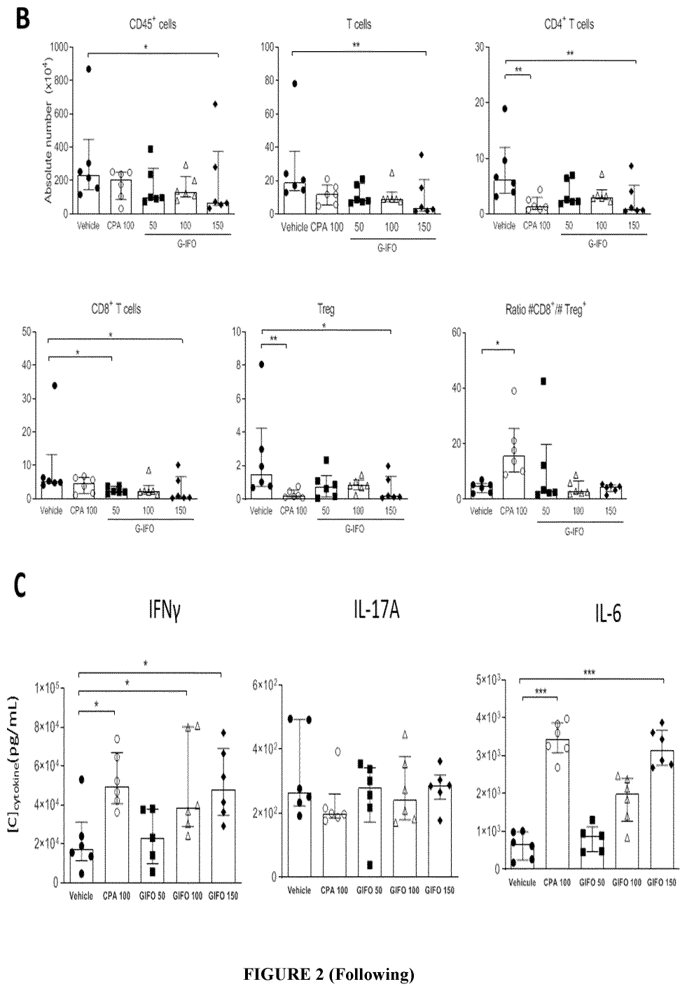

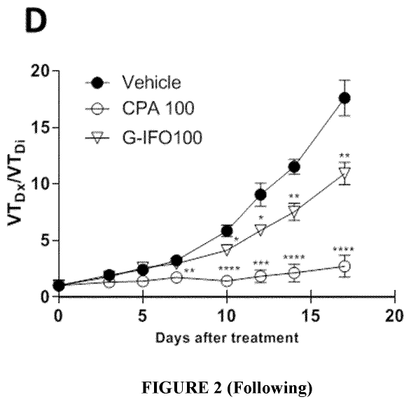

As shown in the Example section, the Inventors demonstrated that oxazaphosphorine derivatives such as geranyloxy-IFO (G-IFO) can display immunomodulatory activity in vivo when used at low doses (FIG. 2). More precisely, the Inventors showed that low dose of G-IFO favored Th1 polarization and induced T cell-dependent antitumor effects in tumor-bearing mice.

Furthermore, the Inventors showed that oxazaphosphorine derivatives of the invention significantly enhanced the efficacy of immune checkpoint immunotherapy in MCA205 tumor model known to be poorly responsive to such immunotherapy.

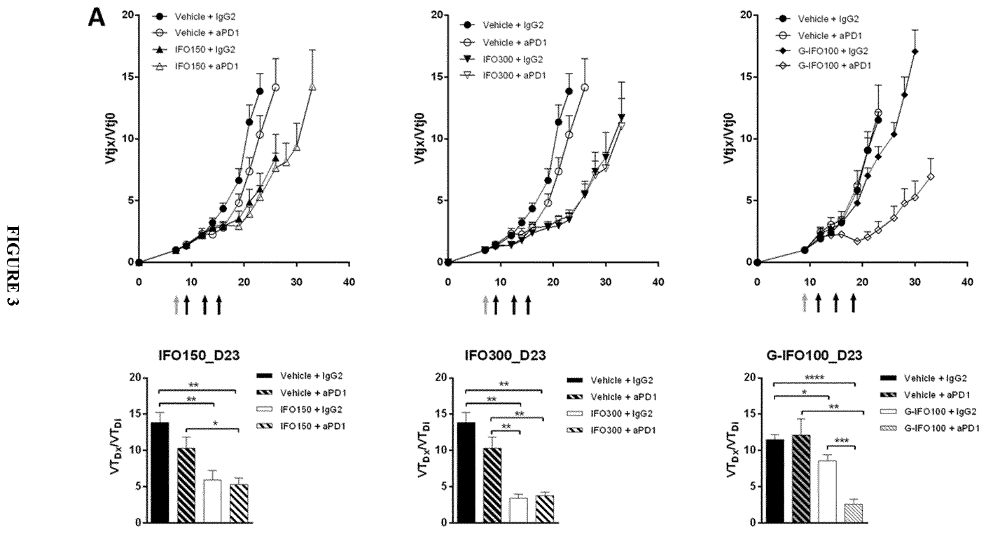

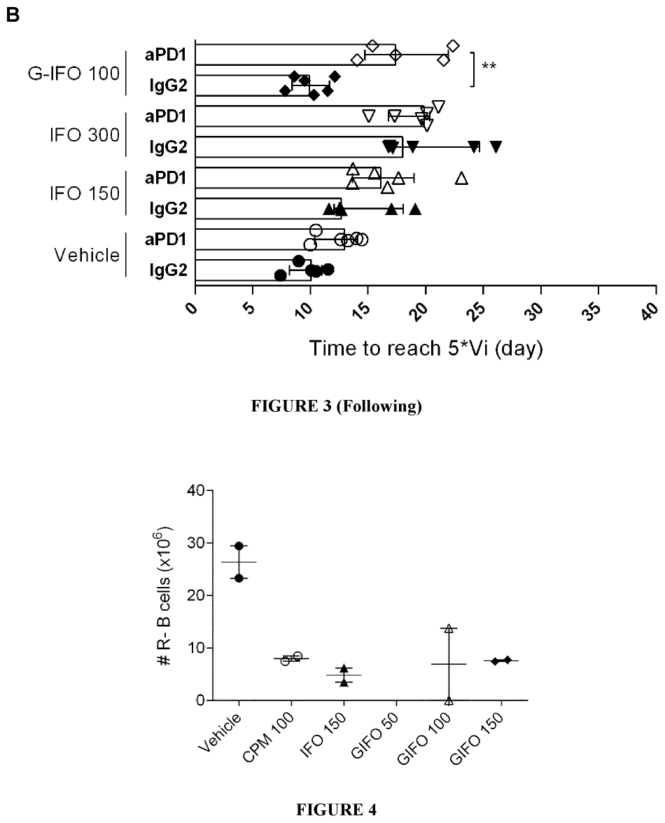

More precisely, the Inventors showed that G-IFO highly decreased tumor growth when used in combination with anti-PD1 mAbs (FIG. 3). Moreover, the time to reach five-fold the initial volume was highly delayed with G-IFO eq. 100 mg/kg+anti-PD1 mAbs compared to G-IFO eq. 100 mg/kg alone and anti-PD1 mAb alone. Altogether, these results clearly demonstrate the synergic effects of G-IFO with anti-PD1 antibodies on tumor growth (FIG. 3B). Of note, such synergic effects were not observed for the therapeutic combination of ifosfamide (IFO) with anti-PD1 antibodies (FIG. 3).

The Inventors further scrutinized immune modifications following i.p. injection of G-IFO in mice. B cells population seemed very affected by oxazaphosphorines even at low dose of G-IFO (eq. 100 mg/kg) underlining high sensitivity of B cells. Such B cells decrease could be an advantage when using an oxazaphosphorine derivative of the invention with an immune checkpoint inhibitor, by preventing or decreasing immune-related adverse events (irAEs) frequently observed with immune checkpoint immunotherapy.

Thus, a first object of the invention is the use of an oxazaphosphorine derivative in combination with an immune checkpoint modulator for preventing or treating a cancer.

The invention also relates to a method for treating or preventing a cancer in a subject, wherein an oxazaphosphorine derivative is administered to the subject in combination with an immune checkpoint modulator.

The invention further relates to the use of an oxazaphosphorine derivative in the preparation of a drug for treating or preventing a cancer, wherein the drug is administered in combination with an immune checkpoint modulator.

The invention also relates to the use of an oxazaphosphorine derivative and an immune checkpoint modulator in the preparation of a drug for treating or preventing cancer.

As used herein, “a combination therapy” or “the use of a drug in combination with another” refers to a treatment in which a subject is administered with two or more therapeutic agents so as to treat a single disease. As described further below, the administration of the two or more therapeutic agents can be performed simultaneously, separately, consecutively, concomitantly, or successively. The effects of the two or more therapeutic agents does not need necessary to produce effects exactly at the same time and/or during the exact same period. The effects of the therapeutic agents need only be overlapping for a period of time sufficient to exert the combined therapeutic activity which is sought by their use in combination.

Thus, a combination therapy does not necessary require the therapeutic agents to be administered at the same time, in a single pharmaceutical composition, in the same pharmaceutical dosage form and/or by the same administration route.

As used herein, the term of “cancer” refers to a disorder in mammals involving upregulated cell growth and characterized by malignancy. The cancer may be of any type. It may be a solid tumor or a hematopoietic cancer.

Preferably, the cancer is selected from the group consisting of carcinoma, sarcoma, lymphoma, leukemia, germ cell tumor, blastoma and melanoma. For instance, the cancer may be selected, without being limited to, chronic myeloid leukemia, acute lymphoblastic leukemia, Philadelphia chromosome positive acute lymphoblastic leukemia (Ph+ ALL), Hodgkin's disease, Hodgkin's and non-Hodgkin lymphomas, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, malignant hepatoma, breast cancer, colon carcinoma, and head and neck cancer, gastric cancer, germ cell tumor, pediatric sarcoma, rhabdomyosarcoma, Ewing's sarcoma, osteosarcoma, soft tissue sarcoma, sinonasal NK/T-cell lymphomas, myelomas, melanomas, Merkel-cell carcinoma (MCC), multiple myeloma, acute myelogenous leukemia (AML), or chronic lymphocytic leukemia.

In some preferred embodiments, the cancer can be selected from the group consisting of the chronic leukemias, acute lymphocytic leukemias, Hodgkin's disease, Hodgkin's and non-Hodgkin lymphomas, cancers of the lung, breast cancer including triple negative breast cancer, genitourinary cancers such as cancers of prostate, bladder, testis, uterine cervix or ovaries, sarcomas such as osteosarcoma and soft tissue sarcoma including pediatric soft tissue sarcoma, neuroblastomas, myelomas, Merkel-cell carcinoma and melanomas.

More preferably, the cancer is selected from sarcomas including osteosarcoma and soft tissue sarcoma, breast cancer including triple negative breast cancer, gastro-intestinal cancer, genitourinary cancers and lung cancer including non-small-cell lung carcinoma and small-cell lung carcinoma.

In some embodiments, the cancer may be a cancer refractory to a previous anticancer therapy such as treatments with chemotherapy, targeted molecular therapy or immunotherapy as standalone or chemotherapy plus immunotherapy treatments. Consequently, the therapeutic combination of the invention is used as a second line-treatment of the cancer in the subject.

In some other embodiments, the therapeutic combination is used as a first-line treatment of the cancer in the subject.

In another embodiment, the cancer may be a relapsed cancer in the subject.

The subject may be a non-human or human, preferably a human being. The subject may be of any gender and/or any age. In some embodiments, the subject is a child. In other embodiment the subject is an adult.

As used herein, “the treatment of cancer” or “treating cancer” includes curing, delaying, alleviating or slowing the progression of the cancer, including that of tumor growth as well as the prevention, the attenuation, the slowing, the reverse or the elimination one or more of the symptoms of the cancer in the subject. It also encompasses the fact of eradicating the tumor in the subject. The term “treatment of a cancer” also encompasses the fact of improving “the overall survival” and/or “the progression free survival” in a subject.

Although not precluded, the wording “treating a cancer” does not mean that the cancer or a symptom associated therewith be completely eliminated in the subject.

An improvement of “the progression free survival” refers to increasing the length of time during and after the treatment of the cancer that the subject lives with the cancer without getting worse. The “overall survival” refers to the length of time from the start of the treatment for the cancer that the patient is still alive. “The progression of free survival” and the “overall survival” figure are typically determined as mean values determined from an appropriate sized clinical trial.

The “prevention of cancer” includes preventing, or delaying the onset of the cancer or one or more symptoms associated with said cancer. The “prevention of cancer” also refers to any act intended to ameliorate the health status of patients such as therapy, prophylaxis and retardation of the disease and/or to prevent the patient from being afflicted by the disease. In some embodiments, this term also refers to minimizing the risk (or the probability) for a patient to develop said cancer, as compared to a patient who has not been administered the therapeutic combination of the invention.

Oxazaphosphorine Derivatives

As used herein, an oxazaphosphorine derivative refers to a compound comprising the moiety (M):

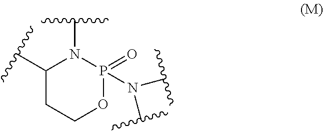 [SMILES: *C1CCOP(=O)(N(*)*)N1*]

In the context of the invention, oxazaphosphorine derivatives of interest are those comprising a substituent at carbon C-4 of the ring. Such oxazaphosphorine derivatives are for instance described in patent applications WO2015/173367 and WO2012/076824, the content of which being incorporated herein by reference.

In the context of the invention, the oxazaphosphorine derivatives of interest are of those of formula (I):

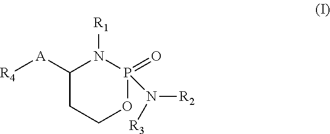

wherein:
A is O—O, O, S, NH, NR₅ with R₅ is an alkyl group, preferably a C₁-C₃ alkyl group, or a linker group preferably having a molecular weight up to 500 g·mol⁻¹, more preferably lower than 400 g·mol⁻¹,
R₁, R₂ and R₃ are independently selected from the group consisting of —H, —CH(CH₃)—CH₂—X and —(CH₂)₂—X, wherein X is an halogen atom, preferably Cl, Br or I, and more preferably Br or Cl,
R₄ is H or a saturated or unsaturated chain of 2 to 30 carbon atoms optionally interrupted by one or several heteroatoms such as S, O and NH, and optionally substituted by one or several substituents independently selected from the group consisting of halogen (e.g. F, Cl, Br, I), CN, CF₃, OH, C₁-C₆ alkyl, C₁-C₆ hydroxyalkyl, C₁-C₆ alkyloxy, C₁-C₆ aminoalkyl, C₁-C₆ halogenoalkyl, —C₂-C₆ alkoxy alkyl, —C(O)OR, —OC(O)R, —OC(O)OR, —C(O)R, —NHC(O)—NH—R, —NH—C(O)—R, —C(O)—NH—R, —NRR′, —C(O)NRR′, —NC(O)R, —NRC(O)R′, and —SR, wherein R and R′ are independently selected from H and C₁-C₆ alkyl, and pharmaceutically acceptable salt or solvate thereof.

As used herein, the term of “pharmaceutically acceptable” refers to compositions, compounds, salts and the like that are, within the scope of sound medical judgment, suitable for contact with the tissues of the subject, or which can be administered to the subject, without excessive toxicity or other complications commensurate with a reasonable benefit/risk ratio.

As used herein, the term “solvate” or “pharmaceutically acceptable solvate” refers to a solvate formed from the association of one or more molecule of compounds of the invention with one or more molecules of solvent. The term solvates include hydrates such as hemi-hydrate, monohydrate, dihydrate, trihydrate, tetrahydrate and the like.

As used herein, the term “pharmaceutically acceptable salt” refers to non-toxic salts, which can generally be prepared by contacting the oxazaphosphorine derivative of the invention with a suitable organic or inorganic acid. For instance, pharmaceutical salts may be, without being limited to, acetate, benzenesulfonate, benzonate, bicarbonate, bisulfate, bitartrate, bromide, butyrate, carbonate, chloride, citrate, diphosphate, fumarate, iodide, lactate, laurate, malate, maleate, mandelate, mesylate, oleate, oxalate, palmitate, phosphate, propionate, succinate, sulfate, tartrate, and the like.

As used herein, a “linker group” refers to any chemical group suitable to connect the R₄ group to the oxazaphosphorine skeleton without impairing the ability of the compound to release the alkylating mustards in vivo. For instance the linker may be selected from the group consisting of:
Natural and non-natural amino acids;
peptides comprising from 2 to 10, preferably from 2 to 5 amino acids and derivatives thereof;
—N(R₆)— with R₆ being an alkyl group, in particular a C₁-C₃ alkyl,
C₁-C₁₀ hydrocarbon chains optionally substituted by one or several substituents selected from —OH, C₁-C₄ alkyl and C₁-C₄ alkyloxy groups, and/or optionally comprising:
   one or several heteroatoms such as NH, S and O; and/or
   one or several chemical groups such as —NHC(O)—, —OC(O)—, OC(O)O, —NH—C(O)—NH—, —S—S—, and —CR₇═N—NH—C(O)—, —ONH—, —ONR₇— —O—C(═S)—S—, —C(═S)—S— wherein R₇ is H or a C₁-C₆ alkyl, and/or
   one or several heteroaryl or aryl groups, and/or
   one or several aliphatic cycles or heterocycles, preferably comprising from 4 to 6 atoms, and optionally substituted by one or several substituents selected from —OH, C₁-C₄ alkyl and C₁-C₄ alkyloxy groups,

In another embodiment, the compound of formula (I) is such that A is selected from the group of O, S, and —NH—, or comprises, or consists of, a spacer moiety selected from the group consisting of:
natural or non-natural amino acids, dipeptides, and derivatives thereof;
polyether groups, such as polyethylene glycol or polypropylene glycol, preferably comprising from 2 to 6 monomers e.g. 2, 3, or 4 monomers;
hydrazone linkers, e.g. of formula —CR₇═N—NH—C(O)— wherein R₇ is H or a C₁-C₆, preferably C₁-C₃ alkyl,
—O—(C═S)—S—, —ONR₇—, —NR₇O—, with R₇ being H or a C₁-C₆ preferably C₁-C₃ alkyl,
Y₁—(CH₂)ₙ—Y_{2,} with n is an integer from 1 to 8, wherein Y₁ and Y₂ are independently selected from —O—, —S—, —OC(O)—, —C(O)O—, —OC(O)—O—, —C(O)NR₇—, NR₇C(O)—, —OC(S)S—, —SC(S)O— —NR₇—, —ONR₇—, —NR₇O—, NR₇C(S)S—, —SC(S)NR₇—
   and

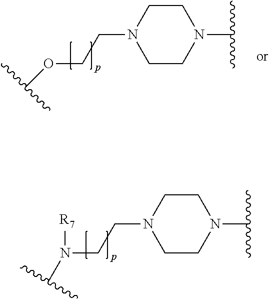

Wherein R₇ is selected from the group of H and C₁-C₆, preferably C₁-C₃ alkyl, and p is an integer from 0 to 8, preferably 1, 2 or 3.

In another embodiment, A consists of, or comprises, a moiety selected from the group consisting of:
O, O—O, S, —O—(C═S)—S—, —ONR₇—, —NR₇O—, with R₇ is H or a C₁-C₃ alkyl, preferably CH₃
Citrulline, lysine, ornithine, alanine, phenylalanine, cysteine, glycine, valine, leucine and dipeptides thereof such as valine-citrulline,
Y₁—(CH₂)ₙ—Y₂, and
Y₁—(CH₂—CH₂—O)ₐ—CH₂—CH₂—Y₂
wherein Y₁ and Y₂ are as defined above, preferably independently selected from O, NR₇, S, OC(O), C(O)O, NHCO, CONH with R₇ is H or a C₁-C₃ alkyl, preferably —CH₃, n is an integer from 1 to 8, preferably 1, 2, 3, or 4 and a is an integer from 1 to 3

Examples of linker of formula Y₁—(CH₂)ₙ—Y₂ encompass cysteamine as well as the following moieties:

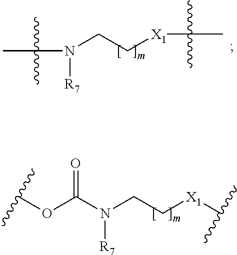

with R₇ is H or a C₁-C₃ alkyl, X₁ is O or S and m is an integer from 0 to 7, preferably 1, 2 or 3.

In some additional embodiments, A is selected from the group consisting of O—O, O, S, —NH-cysteamine linker (i.e. —C(O)NH—CH₂—CH₂—S—), valine-citrulline linker and cysteine linker.

In some embodiments, one of R₁, R₂ and R₃ is H and the two other remaining groups are independently selected from —CH(CH₃)—CH₂—X and —(CH₂)₂—X, with X preferably being Cl or Br.

In an additional embodiment, the compound of formula (I) is such that R₁ is H and R₂ and R₃ are independently selected from —CH(CH₃)—CH₂—X and —(CH₂)₂—X. Preferably R₂ and R₃ are identical.

In an another embodiment, the compound of formula (I) is such that R₂ is H and R₁ and R₃ are independently selected from —CH(CH₃)—CH₂—X and —(CH₂)₂—X. Preferably R₁ and R₃ are identical.

In a particular embodiment, the compound of formula (I) is such that one of R₁, R₂ and R₃ is H and the two other remaining groups are —CH(CH₃)—CH₂—X, with X preferably being Cl or Br.

In another embodiment, the compound of formula (i) is such that one of R₁, R₂ and R₃ is H and the two other remaining groups are —(CH₂)₂—X, with X preferably being Cl or Br.

In a further embodiment, R₁, R₂ and R₃ are independently selected from —CH(CH₃)—CH₂—X and —(CH₂)₂—X. Preferably R₁, R₂ and R₃ are identical.

For instance, R₁, R₂ and R₃ are —CH(CH₃)—CH₂—X with X being preferably Cl or Br.

As another example, R₁, R₂ and R₃ are —CH₂—CH₂—X and —(CH₂)₂—X with X being preferably C₁ or Br.

In some embodiments, R₄ may be H. When R₄ is H, A is preferably O or —O—O—.

Alternatively, R₄ may comprise one or several (e.g. from 1 to 10, e.g. 1, 2, 3, 4 or 5) unsaturations, which can be double and/or triple bonds. In some embodiments, R₄ comprises from 1 to 10, preferably from 1 to 5, double bonds in its backbone.

In some embodiments, R₄ may comprise from 3 to 25 or from 5 to 10 (e.g. 5, 5, 7, 8, 9, 10) carbon atoms.

In some embodiments, R₄ is a saturated or unsaturated, hydrocarbon chain of 2 to 30 carbon atoms optionally substituted by one or several substituents independently selected from the group consisting of halogen (e.g. F, Cl, Br, I), —CN, OH, CF₃, C₁-C₃ alkyl, C₁-C₃ hydroxyalkyl, C₁-C₃ alkyloxy, C₁-C₃ aminoalkyl, C₁-C₃ halogenoalkyl, —C₂-C₄ alkoxy alkyl, —C(O)OR, —OC(O)R, —OC(O)OR, —C(O)R, —NHC(O)—NH—R, —NH—C(O)—R, —C(O)—NH—R, —NRR′, —C(O)NRR′, —NC(O)R, —NRC(O)R′, and —SR, wherein R and R′ are independently selected from H and C₁-C₆ preferably C₁-C₃ alkyl.

In some other embodiments, R₄ is a saturated or unsaturated, hydrocarbon chain of 2 to 30 carbon atoms optionally substituted by one or several substituents independently selected from the group consisting of halogen (e.g. F, Cl, Br, I), —CN, C₁-C₃ alkyl, C₁-C₃ hydroxyalkyl, and C₁-C₃ alkyloxy, preferably from the group consisting of —OH, —F, Cl, Br, I, —OCH₃ and CH₃.

Preferred compounds of formula (I) are those wherein R₄ is selected from the group of unsaturated chains as described above. In some particular embodiments, R₄ is an unsaturated hydrocarbon chain comprising from 3 to 30, preferably from 5 to 30, such as from 5 to 20 or 5 to 10 carbon atoms optionally substituted by one or several substituents independently selected from the group consisting of halogen (e.g. F, Cl, Br, I), —CN, C₁-C₃ alkyl, C₁-C₃ hydroxyalkyl, and C₁-C₃ alkyloxy, preferably from the group consisting of —OH, —F, Cl, Br, I, —OCH₃ and CH₃ and more preferably from —OH, —OCH₃ and —CH₃.

In a particular embodiment of the invention, R₄ comprises one or several isoprene units. For instance, R₄ may consist in, or comprise, an acyclic terpene moiety. For instance, R₄ may comprise, or consist in, a chemical moiety selected from geranyl radical, farnesyl radical and squalenyl radical:

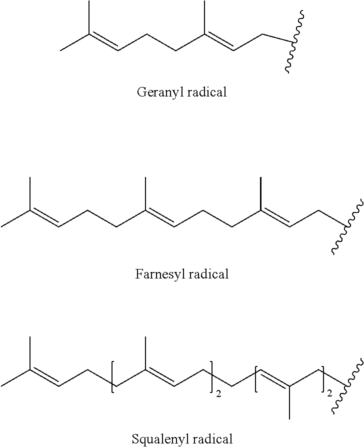

Compounds of interest comprising a squalenyl radical are those described in WO2012/076824, e.g.:

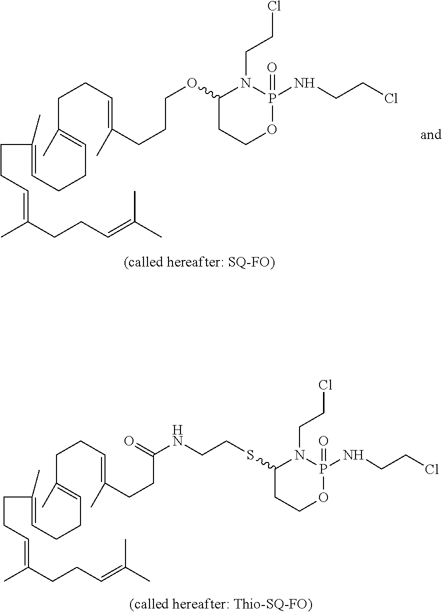 [SMILES: CC(C)=CCC/C(C)=C/CC/C(C)=C/CC/C=C(\C)CC/C=C(\C)CCCOC1CCOP(=O)(NCCCl)N1CCCl ; CC(C)=CCC/C(C)=C/CC/C(C)=C/CC/C=C(\C)CC/C=C(\C)CCC(=O)NCCSC1CCOP(=O)(NCCCl)N1CCCl]

In a preferred aspect, the invention refers to the therapeutic use of an oxazaphosphorine derivative in combination with an immune checkpoint modulator for treating or preventing cancer, wherein the oxazaphosphorine derivative is of formula (Ia):

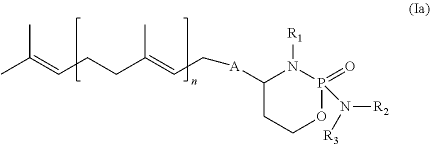

wherein
n is an integer from 0 to 3, preferably 1 or 2,
A, R₁, R₂ and R₃ are as defined for the compound of formula (I)
   and pharmaceutically acceptable salt and solvate thereof.

In some embodiments, the compound of formula (Ia) is such that:
n is 1 or 2,
A is selected from the group of O—O, O, S, and —NH—, preferably O, S and NH or comprises, or consists of, a spacer moiety selected from the group consisting of:
   natural or non-natural amino acids, dipeptides, and derivatives thereof;
   polyether groups, such as polyethylene glycol or polypropylene glycol, preferably comprising from 2 to 6 monomers e.g. 2, 3, or 4 monomers;
   hydrazone linkers, e.g. of formula —CR₇═N—NH—C(O)— wherein R₇ is H or a C₁-C₆, preferably C₁-C₃ alkyl,
—O—(C═S)—S—, —ONR₇—, —NR₇O—, with R₇ being H or a C₁-C₆ preferably C₁-C₃ alkyl,
Y₁—(CH₂)ₙ—Y_{2,} with n is an integer from 1 to 8, wherein Y₁ and Y₂ are independently selected from —O—, —S—, —OC(O)—, —C(O)O—, —OC(O)—O—, —C(O)NR₇—, NR₇C(O)—, —OC(S)S—, —SC(S)O— —NR₇—, —ONR₇—, —NR₇O—, NR₇C(S)S—, —SC(S)NR₇—
and

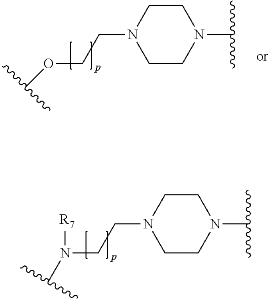

wherein R₇ is selected from the group of H and C₁-C₆, preferably C₁-C₃ alkyl, and p is an integer from 0 to 8, preferably 1, 2 or 3, and
R₁, R₂ and R₃ are such that one of R₁, R₂ and R₃ is H and the two other remaining groups are independently selected from —CH(CH₃)—CH₂—X and —(CH₂)₂—X, with X preferably being Cl or Br. In a particular aspect, the oxazaphosphorine derivative is of formula (Ia) wherein one of R₁, R₂ and R₃ is H and the two other remaining groups are —(CH₂)₂—X, with X preferably being Cl or Br. In another aspect, the oxazaphosphorine derivative is of formula (Ia) wherein one of R₁, R₂ and R₃ is H and the two other remaining groups are —CH(CH₃)—CH₂—X, with X preferably being Cl or Br

In other embodiments, the compound of formula (Ia) is such that:
n is 1 or 2, preferably 1, and
A is O, S or NH, or is a moiety selected from the group consisting of:
   —O—(C═S)—S—, —ONR₇—, —NR₇O—, with R₇ is H or a C₁-C₃ alkyl, preferably CH₃
   Citrulline, lysine, ornithine, alanine, phenylalanine, cysteine, glycine, valine, leucine and dipeptides thereof such as valine-citrulline,
   Y₁—(CH₂)ₙ—Y₂, and
   Y₁—(CH₂—CH₂—O)ₐ—CH₂—CH₂—Y₂
   wherein Y₁ and Y₂ are as defined above, preferably independently selected from O, NR₇, S, OC(O), C(O)O, NHCO, CONH with R₇ is H or a C₁-C₃ alkyl, preferably —CH₃, n is an integer from 1 to 8, preferably 1, 2, 3, or 4 and a is an integer from 1 to 3, and
R₁, R₂ and R₃ are such that one of R₁, R₂ and R₃ is H and the two other remaining groups are identical and selected from —CH(CH₃)—CH₂—X and —(CH₂)₂—X, with X preferably being Cl or Br. In a particular aspect, one of R₁, R₂ and R₃ is H and the two other remaining groups are —(CH₂)₂—X, with X preferably being Cl or Br. In another aspect, one of R₁, R₂ and R₃ is H and the two other remaining groups are —CH(CH₃)—CH₂—X, with X preferably being Cl or Br

In a particular embodiment, the compound of formula (Ia) is such that:
n is 1 or 2, preferably 1,
A is selected from the group consisting of O, S, —NH— cysteamine linker (i.e. —C(O)NH—CH₂—CH₂—S—), valine-citrulline linker and cysteine linker, and
R₁, R₂ and R₃ are such that:
   R₁ is H, and R₂ and R₃ are identical and selected from the group consisting of —CH(CH₃)—CH₂—X and —(CH₂)₂—X with X is Br or Cl; or
   R₂ is H, and R₁ and R₃ are identical and selected from the group consisting of —CH(CH₃)—CH₂—X and —(CH₂)₂—X with X is Br or Cl.

Preferred compounds of formula (Ia) are those of formula (IIa) and (IIb) as shown below:

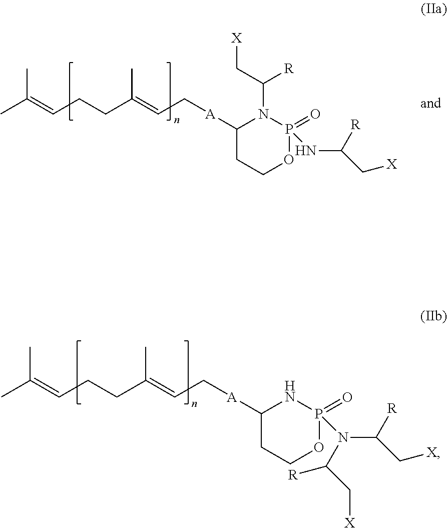

wherein
n is 1 or 2,
R is H or CH₃,
X is Cl or Br, and
A is selected from the group consisting of O, S, —NH—, cysteamine linker, valine-citrulline linker and cysteine linker,
   and pharmaceutically acceptable salts and solvates thereof.

In a particular aspect, the oxazaphosphorine derivative is selected from the group consisting of
A compound of formula (IIa), wherein n is 1, A is O, X is Cl, and R is H,
A compound of formula (IIa), wherein n is 1, A is O, X is Cl, and R is CH₃,
A compound of formula (IIa), wherein n is 1, A is O, X is Br, and R is H,
A compound of formula (IIa), wherein n is 1, A is O, X is Br, and R is CH₃,
A compound of formula (IIa), wherein n is 2, A is O, X is Cl, and R is H,
A compound of formula (IIa), wherein n is 2, A is O, X is Cl, and R is CH₃,
A compound of formula (IIa), wherein n is 2, A is O, X is Br, and R is H,
A compound of formula (IIa), wherein n is 2, A is O, X is Br, and R is CH₃,
A compound of formula (IIb), wherein n is 1, A is O, X is Cl, and R is H,
A compound of formula (IIb), wherein n is 1, A is O, X is Cl, and R is CH₃,
A compound of formula (IIb), wherein n is 1, A is O, X is Br, and R is H,
A compound of formula (IIb), wherein n is 1, A is O, X is Br, and R is CH₃,
A compound of formula (IIb), wherein n is 2, A is O, X is Cl, and R is H,
A compound of formula (IIb), wherein n is 2, A is O, X is Cl, and R is CH₃,
A compound of formula (IIb), wherein n is 2, A is O, X is Br, and R is H,
A compound of formula (IIb), wherein n is 2, A is O, X is Br, and R is CH₃,
   and pharmaceutically acceptable salts and solvates thereof.

For instance, the oxazaphosphorine derivative may be selected from the group consisting of
A compound of formula (IIa), wherein n is 1, A is O, X is Cl, and R is CH₃,
A compound of formula (IIa), wherein n is 1, A is O, X is Br, and R is CH₃,
A compound of formula (IIa), wherein n is 2, A is O, X is Cl, and R is CH₃,
A compound of formula (IIa), wherein n is 2, A is O, X is Br, and R is CH₃,
A compound of formula (IIb), wherein n is 1, A is O, X is Cl, and R is CH₃,
A compound of formula (IIb), wherein n is 1, A is O, X is Br, and R is CH₃,
A compound of formula (IIb), wherein n is 2, A is O, X is Cl, and R is CH₃,
A compound of formula (IIb), wherein n is 2, A is O, X is Br, and R is CH₃,
   and pharmaceutically acceptable salts and solvates thereof.

As another example, the oxazaphosphorine derivative is selected from the group consisting of
A compound of formula (IIa), wherein n is 1, A is O, X is Cl, and R is H,
A compound of formula (IIa), wherein n is 1, A is O, X is Br, and R is H,
A compound of formula (IIa), wherein n is 2, A is O, X is Cl, and R is H,
A compound of formula (IIa), wherein n is 2, A is O, X is Br, and R is H,
A compound of formula (IIb), wherein n is 1, A is O, X is Cl, and R is H,
A compound of formula (IIb), wherein n is 1, A is O, X is Br, and R is H,
A compound of formula (IIb), wherein n is 2, A is O, X is Cl, and R is H,
A compound of formula (IIb), wherein n is 2, A is O, X is Br, and R is H,
   and pharmaceutically acceptable salts and solvates thereof.

In a more particular embodiment, the oxazaphosphorine derivative is selected from:
A compound of formula (IIa), wherein n is 1, A is O, X is Cl, and R₄ is H,

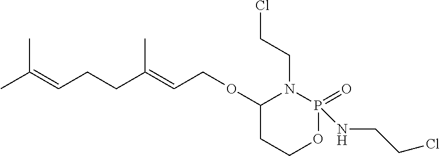 [SMILES: CC(C)=CCC/C(C)=C/COC1CCOP(=O)(NCCCl)N1CCCl]

(hereafter called: geranyloxy-IFO),
A compound of formula (IIa), wherein n is 1, A is O, X is Br, and R₄ is CH₃,

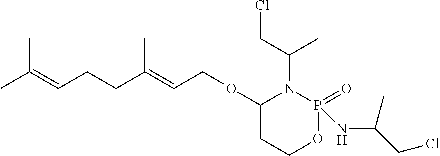 [SMILES: CC(C)=CCC/C(C)=C/COC1CCOP(=O)(NC(C)CCl)N1C(C)CCl]

(hereafter: methylated geranyloxy-IFO),

and pharmaceutically acceptable salts and solvates thereof.

In another embodiment, the oxazaphosphorine derivative is selected from:
A compound of formula (IIb), wherein n is 1, A is O, X is Cl, and R₄ is H,

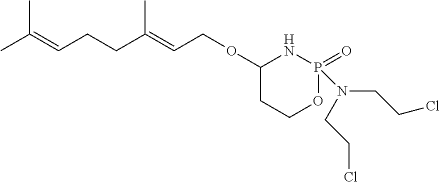 [SMILES: CC(C)=CCC/C(C)=C/COC1CCOP(=O)(N(CCCl)CCCl)N1]

(hereafter called: geranyloxy-CPA),
A compound of formula (IIb), wherein n is 1, A is O, X is Br, and R₄ is CH₃,

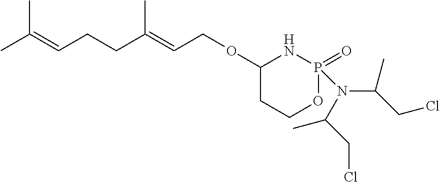 [SMILES: CC(C)=CCC/C(C)=C/COC1CCOP(=O)(N(C(C)CCl)C(C)CCl)N1]

(hereafter: methylated geranyloxy-CPA),
and pharmaceutically acceptable salts and solvates thereof.

Methods for preparing a compound of formula (I), (Ia), (IIa) and (IIb) are well-known. The skilled artisan may refer to standard procedures. The skilled artisan may refer to any one of the synthetic methods described in patent application WO2012/076824 and WO2015/173367.

Certain oxazaphosphorine derivatives according to the invention, in particular those bearing a linear terpene moieties (e.g. farnesyl, squalenyl and geranyl radicals) at position C-4, and preferably compounds of formula (Ia), are able to self-organize into nanoparticles. Said self-assembly into nanoparticles may increase the biological activity of the compound such as its cytotoxicity and improve its delivery to cancerous cells. Moreover, the compound in nanoparticle form may have an improved stability as compared to its free form under storage. In some embodiments, the oxazaphosphorine derivative is in the form of nanoparticles.

Thus, in a particular embodiment of the invention, the oxazaphosphorine derivative is administered to the patient in the form of nanoparticles. In such an embodiment, the oxazaphosphorine derivative is present as a constituent, more preferably as the main component of the nanoparticle, which means that the oxazaphosphorine derivative on may account for more than 50% in weight, e.g. more than 60%, 70%, 80%, 90%, 95%, 98%, 99%, or 99.5% in weight of the total weight of the nanoparticle. In some embodiments, the nanoparticle is formed by self-organization of molecules of an oxazaphosphorine derivative of formula (I), preferably of formula (Ia) such as those of formula (IIa) and (IIb).

The mean hydrodynamic diameter of such nanoparticle of the invention is typically from 10 to 800 nm, preferably from 30 to 500 nm and in particular from 50 to 400 nm. For instance, the nanoparticles may have a mean hydrodynamic diameter from 70 nm to 200 nm, for instance from 100 nm to 250 nm. The mean hydrodynamic diameter is preferably determined by Dynamic Light scattering at 20° C., for instance by using a Nanosizer ZS (Malvern Instrument Ltd, France). The nanoparticles of oxazaphosphorine derivatives can be obtained by dissolving the derivative in an organic solvent such as acetone or ethanol, then adding this mixture into an aqueous phase under stirring leading to the formation of nanoparticles with or without surfactant(s). Surfactants include, for example, polyoxyethylene-polyoxypropylene copolymers, sodium lauryl sulfate, phospholipid derivatives and lipophilic derivatives of polyethylene glycol.

In some embodiments, the oxazaphosphorine derivative is administered in the form of a nanoparticles present in a colloidal system, preferably in an aqueous medium to the subject.

Immune Checkpoint Modulators

As used herein, “immune checkpoint pathways” refer to regulator pathways of the immune system that are responsible for maintaining self-tolerance and modulating the duration and the amplitude of the immune response.

It was shown that tumor cells can evade immunosurveillance by activation of immune checkpoint pathways that suppress antitumor immune response. In particular, it was shown that tumor cells may provide mediators of immune escape though the expression of ligands, such as PD-L1, for immune checkpoints establishing an immune tolerant landscape. Immune checkpoint modulators such as anti-PD-1, anti-PD-L1 and anti-CTLA-4 monoclonal antibodies were shown to be effective towards many tumors such as melanoma and small-cell lung cancer (Hodi, NEJM, 2010, 363(8):711-723, Zielinski, Annals of Oncology, 2013, 24(5):1170-9).

In the context of the invention, an immune checkpoint modulator refers to any therapeutic agent able to prevent or decrease the evasion of tumor cells from immunosurveillance, e.g. able to induce, enhance, sustain, restore, activate or, prevent the deactivation of, an immune response, preferably an anti-tumor response, more preferably a T-cell-mediated anti-tumor immune response.

Immune checkpoint pathways are regulated by immune checkpoint proteins.

As used herein, an “immune checkpoint protein” is a protein, typically a receptor (e.g., CTLA4 or PD-1) or a ligand (e.g., PD-L1 or PD-L2) that regulates or modulates the extent of an immune response. The immune checkpoint proteins can be inhibitory or stimulatory. In particular, the immune checkpoint proteins may be inhibitory to the activation of the immune response. Thus, inhibition of an inhibitory immune checkpoint protein acts to stimulate or activate an immune response, such as T cell activation and proliferation. Similarly, activation of a stimulatory immune checkpoint protein acts to stimulate or activate an immune response.

In the context of the invention, target immune checkpoint proteins encompass, without being limited to, PD1 (Programmed Death-1) and its ligand PD-L1 and PD-L2, CTLA4 (cytotoxic T-lymphocyte antigen-4), LAG-3, TIM-3, TIGIT, 2B4/CD244 as well as OX40.

In some embodiments, the immune checkpoint modulator refers to a therapeutic agent able to inhibit or block an inhibitory immune checkpoint pathway. In such a case, the immune checkpoint modulator is an immune checkpoint inhibitor, also known as immune checkpoint blockers (ICB).

In some particular embodiments, the immune checkpoint inhibitor is an agent which inhibits CTLA-4, PD-1, LAG-3, TIM-3, TIGIT or 2B4/CD244 immune checkpoint pathways. In some preferred embodiments, the targeted proteins are selected from LAG-3, PD1, ligand PD-L1, CTLA, TIM-3, TIGIT and 2B4/CD244. In some other embodiments, the immune checkpoint inhibitor is selected from PD1 inhibitors, PD-L1 inhibitors, CTLA-4 inhibitors, LAG-3 inhibitors, TIM-3 inhibitors, TIGIT inhibitors, 2B4/CD244 inhibitors and combinations thereof, more preferably from PD1 inhibitors, PD-L1 inhibitors, CTLA-4 inhibitors, TIGIT inhibitors and combinations thereof, and even more preferably from PD1 inhibitors, PD-L1 inhibitors, and CTLA-4 inhibitors.

As used herein, an inhibitor refers to a therapeutic agent able to bind to a protein target of interest and inhibits (e.g. blocks or decreases) its activity. For instance, an immune checkpoint inhibitor may block an inhibitory pathway by binding and/or blocking an immune checkpoint protein of interest. The inhibitor may be competitive or non-competitive, e.g. steric or allosteric.

In some other embodiments, the immune checkpoint modulator refers to a therapeutic agent able to activate a stimulatory immune checkpoint pathway, e.g. by binding and activating a stimulatory immune checkpoint receptor. For instance, such an immune checkpoint modulator is an agent which activates OX40 immune pathway, typically an agonist of OX40 receptor.

In a particular embodiment, the immune checkpoint modulator is selected from the group consisting of CTLA-4 inhibitors, PD-1 inhibitors, PD-L1 inhibitors, TIGIT inhibitors, LAG-3 inhibitors, TIM-3 inhibitors, OX-40 agonists and combinations thereof.

In the context of the invention, the immune checkpoint modulator can be of any type and can be selected from small synthetic chemical drugs, proteins such as ligands and fusion proteins, antibodies and fragments thereof, peptides and nucleic acids such as aptamers and antisense oligonucleotides.

As used herein, an “aptamer” (also called nucleic aptamer) refers to a synthetic single-stranded polynucleotide typically comprising from 20 to 150 nucleotides in length and able to bind with high affinity a target molecule. The aptamers are characterized by three-dimensional conformation(s) which may play a key role in their interactions with their target molecule.

An “Antisense oligonucleotide” refers to a nucleic acid capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, meroduplex (mdRNA) and satellite repeats.

As used herein, the term “antibody” refers to an immunoglobulin or a fragment or a derivative thereof, and encompasses any polypeptide comprising an antigen-binding domain, regardless whether it is produced in vitro or in vivo. The term includes, but is not limited to, polyclonal, monoclonal, monospecific, multispecific (e.g. bispecific), humanized, single-chain, chimeric, synthetic, recombinant, hybrid, mutated, and grafted antibodies. The term “antibody” also includes antibody fragments such as Fab, F(ab′)2, Fv, scFv, Fd, dAb, and other antibody fragments (e.g. VHH from single-chain antibody) that retain antigen-binding function, i.e., the ability to bind their target specifically. Typically, such fragments would comprise an antigen-binding domain.

The terms “antigen-binding domain,” or “antigen-binding fragment,” refer to a part of an antibody molecule that comprises amino acids responsible for the specific binding between the antibody and the antigen. Where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain may comprise an antibody light chain variable region (VL) and an antibody heavy chain variable region (VH). However, it does not necessarily comprise both (see e.g. the antigen-binding domain of single chain antibodies and VHH fragments). Typically, an antigen-binding fragment or domain contains at least a portion of the variable regions (heavy and light) of the antibody sufficient to form an antigen binding site (e.g., one or more CDRs, and generally all CDRs) and thus retains the binding specificity and/or activity of the anti-immune checkpoint protein antibody.

Variants of antibodies directed against a protein target of immune checkpoint pathways (e.g., anti-CTLA-4, anti-PD-L1, anti-TIGIT, anti-LAG3, anti-TIM-3 or anti-PD-1 antibodies) are also included in the invention with proviso that they retain the ability to specifically bind their target and exert the requested action on this target (e.g. inhibits or blocks the target). Such variants may be derived from the sequence of antibodies described in the prior art by using routine techniques.

A variant differs of its parent polypeptide in virtue of one or several (e.g. 1, 2, 3, 4, 5, 10, 20, 30, 40, 50 60, or more) amino acid mutations. Amino acid mutations encompass the substitution of an amino acid, the deletion of an amino acid of the addition of the amino acid. As used herein, a variant of a parent polypeptide also encompass polypeptides which differ from its parent polypeptide in virtue of one or several glycosylation modifications. A polypeptide variant may have at least 70%, preferably at least 75%, 80%, 85%, 90%, 95%, 97%, 98%, of 99% of amino acid sequence identity with its parent polypeptide.

For example, in the case of antibody variants, amino acid substitutions, deletions, or additions, can be made in the FRs (frameworks) and/or in the CDRs (Complementarity Determining Regions) of the antibody of interest. While changes in the FRs are usually designed to improve stability and immunogenicity of the antibody, changes in the CDRs are typically designed to increase affinity of the antibody for its target. As another example, amino acid mutations can be introduced in the Fc region of the antibody so as to increase its half-life (and thus decrease its clearance) or modulate its effector function, if any, (e.g. increase or decrease complement dependent cytotoxicity (CDC), antibody dependent cytotoxicity (ADCC) or antibody dependent cellular phagocytosis (ADCP)). Derivatives and variants of antibodies of the invention can be produced by various techniques well known in the art, including recombinant and synthetic methods, analogous shuffling or combinatorial techniques, mutagenesis including random mutagenesis and the like.

The antibodies for use in the invention can be linked to another functional molecule, e.g., another peptide or protein (albumin, another antigen binding domain, etc.), a drug, a ligand for enhancing its delivery to the tumor, a radionuclide or a label such as a fluorescent label. The antibodies may also be coupled to synthetic polymers, e.g., polyethylene glycol for example, in order to increase their circulating half-life. The antibodies may also have an altered glycosylation pattern, e.g. one or more carbohydrate moieties may be deleted and/or one or more glycosylation sites added to the original antibody.

In some embodiments, the immune checkpoint modulator is a full-length antibody, preferably monoclonal full-length antibody, a variant thereof or a binding-domain fragment thereof as well as biosimilar antibodies thereof.

As used herein, a “full-length antibody” (also called herein immunoglobulin of Ig) refers to a protein having the structure that constitutes the natural biological form of an antibody, including variable and constant regions. “Full length antibody” covers both monoclonal and polyclonal full-length antibodies and also encompasses wild-type full-length antibodies, chimeric full-length antibodies, humanized full-length antibodies, the list not being limitative. In most mammals, including humans and mice, the structure of full-length antibodies is generally a tetramer. Said tetramer is composed of two identical pairs of polypeptide chains, each pair having one “light” (typically having a molecular weight of about 25 kDa) and one “heavy” chain (typically having a molecular weight of about 50-70 kDa). In the case of human immunoglobulins, light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. Thus, “isotype” as used herein is meant any of the classes of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE.

In some embodiments, the immune checkpoint modulator is selected from the group constituting of fully-human Igs, humanized Igs, chimeric Igs and variants thereof, preferably of isotype IgG or IgA, e.g. IgG1, IgG2, IgG3 and IgG4 and variants thereof.

“An aptamer/antibody specifically binds to the immune checkpoint target” means that the aptamer or the antibody displays a high affinity for the target molecule. The dissociation constant (Kd) of the aptamer or the antibody for its target molecule is typically lower than 10⁻⁶ M, preferably lower than 10⁻⁸ M, e.g. from 10⁻⁹ to 10⁻¹² M. The term “specifically binding” is used herein to indicate that the aptamer or the antibody has the capacity to recognize and interact specifically with its target in vitro, while having relatively little detectable reactivity with other molecules which may be present in the sample. Kd can be determined by any conventional method, for instance by ELISA-type assay or by surface plasmon resonance (SPR).

In some embodiments, the immune checkpoint modulator is an immune checkpoint inhibitor targeting PD1, PD-L1, TIGIT or CTLA4, more preferably PD1, PD-L1 or CTLA4. Such inhibitors, especially of the antibody type, are described in the literature, some of them being under clinical trial and even already approved in the treatment of certain cancers. For a review about this matter, one can refer to Darvin et al., Experimental & Molecular Medecine (2018) 50:165, the content of which being incorporated herein by reference.

In some preferred embodiments, the immune checkpoint inhibitor is selected from the group consisting of anti-PD1 antibodies, anti-PD-L1 antibodies, anti-CTLA4 antibodies, and combinations thereof.

In a particular embodiment, the immune checkpoint inhibitor is a therapeutic combination itself wherein the first therapeutic agent is selected from anti-PD1 antibodies and anti-PD-L1 antibodies and the second therapeutic agent is selected from anti-CTLA4 antibodies. Another example of therapeutic combination of interest comprises an anti-PD-L1 antibody and an anti-TIGIT antibody.

a) Immune checkpoint inhibitors directed against PD1 or its ligand PD-L1 or PD-L2

PD1 is also known as PDCD1, CD279, PD-1, PD1, hPD-1, SLEB2h, SLE1 and Programmed cell death 1. PD1 is a transmembrane cell surface receptor of the immunoglobulin (Ig) superfamily expressed on activated T cells and pro-B-cells. PD1 negatively regulates T-cell activation and effector function when activated by its ligands. In human, PD1 is encoded by PDCD1 gene (NCBI Gene ID: 5133) and preferably has the sequence set forth in Uniprot database (e.g. Uniprot accession No. Q15116.3 of Apr. 17, 2007). Its endogenous ligands encompass PD-L1 and PD-L2

PD-L1 is also known as CD274, B7-H; B7H1; PDL1; hPD-L1; PDCD1L1 or PDCD1LG. PD-L1 is an immune inhibitory receptor ligand that is expressed by hematopoietic and non-hematopoietic cells, such as T cells and B cells and various types of tumor cells. The encoded protein is a type I transmembrane protein that has immunoglobulin V-like and C-like domains. Interaction of this ligand with its receptor PD1 inhibits T-cell activation and cytokine production. In humans, PD1 is encoded by CD274 gene (NCBI Gene ID: 29126) and preferably has the sequence set forth in Uniprot database (e.g. Uniprot accession No. Q9NZQ7.1 of Oct. 1, 2000).

PD-L2 also known as Programmed cell death 1 ligand 2, B7DC, or PDL2, belongs to immunoglobulin superfamily and is involved in the costimulatory signal essential for T-cell proliferation and IFNG production in a PD1-independent manner. PD-L2 is encoded by PDCD1LG2 gene (NCBI Gene ID: 80380) in humans and preferably has the sequence set forth in Uniprot database (e.g. Uniprot accession No. Q9WUL5.1 of Nov. 1, 1999).

PD1 and its endogenous ligands are immune checkpoint proteins of interest as they play an important role in tumor evasion from host immunity. These cell surface-bound ligand-receptor pairs dampen immune responses to prevent an over-reaction of the immune system. During infection or inflammation of normal tissue, the interaction between PD1 and its ligands is important for preventing autoimmunity by maintaining homeostasis of the immune response through the inhibition of T-cell activation and cytokine production. In tumor microenvironments, this interaction provides an immune escape for tumor cells through cytotoxic T-cell inactivation. It was shown that cancer cells often hijack the normal PD-L1-PD1 immune checkpoint mechanism by overexpressing the ligand PD-L1, which binds to PD1 on effector CD8 T cells, thereby preventing the T cells from mounting an immune response to the tumor. PD-L1 was shown to be expressed in a broad range of cancers.

In some embodiments of the invention, the immune checkpoint inhibitor targets PD1 and/or PDL-1. It was shown that antibodies that block the binding between PD1 with its ligand PD-L1 and/or PD-L2 prevent the activation of PD-1 and its downstream signaling pathways. Such antibodies may restore immune function through the activation of both T cells and T-cell-mediated immune responses against tumor cells.

Accordingly, in some embodiments, the immune checkpoint inhibitor is an entity able to prevent the binding of receptor PD1 to its ligand PD-L1 and/or PD-L2. In some other or additional embodiments, said immune checkpoint inhibitor is selected from antagonists of PD-L1, PD-L2, or PD1. In a particular embodiments, the immune checkpoint inhibitor is selected from the group consisting of anti-PD-L1 aptamers, anti-PD1 aptamers, anti-PD-L1 antibodies and anti-PD1 antibodies, preferably from blocking anti-PD1 or anti-PDL1 antibodies.

As used herein, a “anti-PD-L1 antibody”, “anti-PD1 antibody”, or “anti-PD-L2 antibody”, refers to an antibody which selectively binds to PD-L1 polypeptide, PD1 polypeptide or PD-L2 polypeptide respectively or a soluble fragment thereof. Preferably, said antibody is able to prevent, more precisely to block, the binding of PD1 to its ligand PD-L1 and/or PD-L2. In other words, said antibody is preferably a blocking antibody.

In some embodiments, the anti-PD1 and anti-PD-L1 antibodies are monoclonal antibodies e.g. fully-human, humanized, grafted or chimeric monoclonal antibodies and antigen-binding fragments thereof.

Anti-PD1 and anti-PD-L1 antibodies are described in the prior art, see for instance the following patent applications and patents WO2011066389, WO200705874, WO200114556, US20110271358, U.S. Pat. No. 8,217,149, US20120039906, US20140044738, U.S. Pat. No. 8,779,108, WO200989149 and EP3209778.

Several anti-PD1 and anti-PD-L1 antibodies have been approved for the treatment of certain cancers, especially refractory and relapsed cancers, or are under clinical trial (Darvin et al., 2018, -supra).

Examples of anti-PD1 antibodies encompass, without being limited to, pembrolizumab (Keytruda®), nivolumab (Opdivo®), REGN2810 (also known as cemiplimab), Camrelizumab (also known as SHR-1210), sintilimab (also known as IBI308—Tyvyt®), spartalizumab (PDR001), Tislelizumab (also known as BGB-A317), pidilizumab and JS001.

Examples of anti-PD-L1 antibodies encompass, without being limited to, avelumab (Bavencio®), atezolizumab (Tecentriq®), durvalumab (Imfinzi®), BMS936559, MDX-1105, and KN305.

The immune checkpoint inhibitor may be selected from the hereabove cited anti-PD1 antibodies and anti-PD-L1 antibodies as well as variants thereof, binding-domain fragments thereof and biosimilars thereof.

b) Immune Checkpoint Inhibitors Directed Against CTLA-4

CTLA-4 is also known as CD; GSE; GRD4; ALPS5; CD152; CTLA-4; IDDM12; CELIAC3 and cytotoxic T-lymphocyte associated protein 4. In humans, CTLA-4 is encoded by CTLA4 gene (NCBI gene ID: 1493) and may have the amino acid sequence as shown in Uniprot database e.g. under the accession number P16410.3 (dated Jan. 10, 2003).

CTLA-4 is a member of immunoglobulin superfamily which comprises a V domain, a transmembrane domain, and a cytoplasmic tail. CTLA-4 is expressed in regulatory T cells (Treg), T helper cells and CD8+ T cells. CTLA-4 was shown to regulate the amplitude of the early activation of naive and memory T cells following TCR engagement and to be part of a central inhibitory pathway that affects both antitumor immunity and autoimmunity. The expression of its ligands CD80 (B7.1) and CD86 (B7.2) is largely restricted to antigen-presenting cells, T cells, and other immune mediating cells. CTLA-4 binds CD80 and CD86 with higher affinity than CD28 thus enabling it to outcompete CD28 for its ligands and transmit an inhibitory signal to T cells.

Blocking anti-CTLA-4 antibodies have been reported to enhance T cell activation.

Several anti-CLA4 antibodies are described in the prior art. One can refer, for instance, to patent application WO00/37504 and EP3209778.

In some embodiments, the anti-CTLA4 antibody is selected from monoclonal antibodies e.g. fully-human, humanized, grafted or chimeric monoclonal antibodies and antigen-binding fragments thereof.

Several anti-CTLA-4 antibodies have been approved for the treatment of certain cancers, especially refractory and relapsed cancers, or are under clinical trial (Darvin et al., 2018, -supra).

Examples of anti-CTLA-4 antibodies encompass, without being limited to, Ipilimumab (Yervoy®), tremelimumab, Fc-engineered IgG1 anti-CTLA-4 human monoclonal antibody (AGEN1181) (Agenus Inc.) and non-fucosylated anti-CTLA-4 (BMS-986218).

In some embodiments of the invention, the immune checkpoint inhibitor is selected from Ipilimumab, tremelimumab, variants thereof, biosimilar antibodies thereof, antigen-binding fragments thereof and combinations thereof.

Other immune checkpoint inhibitors of interest encompass bispecific antibodies directed against CTLA4 and PD1. Such bispecific antibodies are for instance described in patent application WO2018/036473.

c) Other Immune Checkpoint Targets of Interest

Several other immune checkpoint targets have been identified, among them LAG3 (lymphocyte activation gene 3), TIM-3, VISTA, TIGIT, 2B4/CD244 as well as costimulatory molecules including ICOS, OX40 and 4-1BB (Sharma and Allison, Cell, 2015, 161, 205-2014).

Thus, the immune checkpoint modulators of interest may be selected from LAG3 inhibitors, TIM-3 inhibitors, VISTA inhibitors, TIGIT inhibitors, 2B4/CD244 inhibitors, ICOS agonists, OX40 agonists, and 4-1B agonists.

Such therapeutic agents are described in the prior art (for instance, see Sharma and Allison, supra).

Among these targets, LAG3, TIM-3, TIGIT and OX-40 are of a particular interest.

The term “LAG3” or “Lymphocyte Activation Gene 3” or CD223 is a type I transmembrane protein that is expressed on the cell surface of activated CD4⁺ and CD8⁺ T cells and subsets of NK and dendritic cells (Triebel F, et al., J. Exp. Med. 1990; 171:1393-1405; Workman C T, et al., J. Immunol. 2009; 182(4):1885-91). LAG3 has 4 extracellular Ig-like domains and require binding to their ligand, major histocompatibility complex (MHC) class II, for their functional activity. The amino acid sequence of an exemplary human LAG3 may be found under UniProt Accession Number P18627. Several anti-LAG3 antibodies are described in the state in the art, for instance in WO1991/10682 and WO2015/138920.

Examples of anti-LAG3 antibody, we can cite relatlimab (BMS) which is under clinical trial assessment, IMP321 or Eftilagimod alpha (Immutep), GSK2831781 (GSK), BMS-986016 (BMS), Ieramilimab or LAG525 (Novartis), REGN3767 (Regeneron Pharmaceuticals).

The term “TIM-3” or “T-cell immunoglobulin mucin-3” also known as HAVCR2, is an important cancer immune checkpoint. TIM-3 is detected in different types of immune cells, including T cells, regulatory T cells (Tregs), dendritic cells (DCs), B cells, macrophages, nature killer (NK) cells, and mast cell. It is a type I membrane protein and consists of 281 amino acids.

The amino acid sequence of an exemplary human TIM-3 may be found under UniProt Accession Number Q8TDQ0. Several anti-TIM-3 antibodies are described in the state in the art, for instance in He et al. (Onco Targets Ther. 2018; 11:7005-7009), Das et al. (Immunol. Rev. 2017; 276(1): 97-111).

Examples of anti-TIM-3 antibody, we can cite MBG453 (Novartis), TSR-022 (Tesaro), LY3321367 (Ely Lilly), MBG453 (Novartis).

The term “TIGIT” or “T-cell immunoreceptor with Ig and ITIM domains” is an immunomodulatory receptor expressed primarily on activated T cells and NK cells. TIGIT's role in tumor immunosurveillance is analogous to the PD-1/PD-L1 axis in tumor immunosuppression. Its structure shows one extracellular immunoglobulin domain, a type 1 transmembrane region and two ITIM motifs. The amino acid sequence of an exemplary human TIGIT may be found under UniProt Accession Number Q495A1. Several anti-TIGIT antibodies are described in the state in the art, for instance in WO2017/053748, WO2017/030823 Examples of anti-TIGIT antibody, we can cite tiragulomab (Roche) which is under clinical trial assessment, Monoclonal Antibody BMS-986207 (BMS); Vibostolimab (MK-7684); OMP-313M32 (OncoMed Pharmaceuticals, Inc.); MTIG7192A (Genentech, Inc.); BGB-A1217 (BeiGene).

In certain embodiments of the invention, the immune modulator checkpoint is selected from blocking anti-LAG3 antibodies, blocking anti-TIM-3 antibodies and blocking anti-TIGIT antibodies, preferably selected from the above-cited antibodies.

OX40 is also known as TNF receptor superfamily member 4, ACT35; CD134; IMD16; and TXGP1L. In humans, OX40 is encoded by TNFRSF4 gene (NCBI gene ID: 7293) and may have the amino acid sequence as shown in Uniprot database under the accession number P47741.1 (dated Feb. 1, 1996). OX40 is a tumor necrosis factor receptor (TNFR) found primarily on activated CD4+ and CD8+ T cells, regulatory T cells (Treg), and natural killer (NK) cells. Signaling through OX40 on activated CD4+ and CD8+ T cells leads to enhanced cytokine production, granzyme and perforin release, and expansion of effector and memory T-cell pools. In addition, OX40 signaling on Treg cells inhibits expansion of Tregs, shuts down the induction of Tregs, and blocks Treg-suppressive function. Thus, agonists of OX40 receptor may be used as immune checkpoint modulators according to the invention. As used herein, an agonist of OX40 receptor refers to any therapeutic agent able to activate OX40 receptor. Agonists of OX40 encompass partial and full agonists and may be of any type, including the entire OX40 ligand (also known as OX401, Tumor necrosis factor ligand superfamily member 4), soluble OX401 as well as variants, fusion proteins and fragments thereof. OX401 may have the amino acid sequence shown in Uniprot database under the accession number P43488.1. Fusion proteins, variants and fragments of OX401 encompassed in the invention are thus capable to bind and activate OX40. Fusion proteins of interest are for instance described in patent application WO2006121810 and U.S. Pat. Nos. 7,959,925 and 6,312,700. As an example, the OX40 agonist may be the fusion protein termed as MEDI6383 in which OX40L is fused with IgG4P Fc.

Agonists of OX40 receptor of interest also encompass agonist aptamers and agonist antibodies. An agonist OX40 aptamer or antibody refers to an aptamer or an antibody respectively able to specifically bind and activate OX40.

Agonist OX40 aptamers are described for instance in Dollis, Chem Biol. 2008 Jul. 21; 15(7): 675-682.

Agonist OX40 antibodies are also described in the prior art, e. g. in Weinberg et al. J Immuother, 2006, 26, 575-585, Morris, 2007, Mol Immunol, 44(2), 3112-3121 or in EP3209778.

In some embodiments, the agonist OX40 antibody is selected from monoclonal antibodies e.g. fully-human, humanized, grafted or chimeric monoclonal antibodies and antigen-binding fragments.

For instance, GSK3174998 is an agonist OX40 antibody currently under clinical trial as a single agent or in combination with Keytruda® in the treatment of different type of cancers. Other examples of anti-OX40 antibodies are MEDI6469 (Medimmune) and BMS 986178 (BMS).

Examples of Therapeutic Combinations of the Invention

In some particular aspects, the invention relates to an oxazaphosphorine derivative in combination with an immune checkpoint modulator for use in treating or preventing a cancer wherein:
the oxazaphosphorine derivative is of formula (Ia) as described above, and
the immune checkpoint modulator is selected from the group consisting of PDL1 inhibitors, PD1 inhibitors, CTLA4 inhibitors, TIGIT inhibitors, LAG-3 inhibitors, TIM-3 inhibitors, OX40 agonists and combinations thereof.

In another particular embodiments, the invention relates to an oxazaphosphorine derivative for use in combination with an immune checkpoint inhibitor for treating or preventing a cancer wherein:
the oxazaphosphorine derivative is of formula (IIa) or (IIb) as described above, and
the immune checkpoint inhibitor is selected from the group consisting of PDL1 inhibitors, PD1 inhibitors, and CTLA4 inhibitors, and combinations thereof, preferably PDL1 inhibitors, PD1 inhibitors and combinations thereof.

For instance, the oxazaphosphorine derivative may be selected from compounds of formula (IIa) or (IIb) wherein n is 1 and/or the immune checkpoint inhibitors may be selected from anti-PD1 antibodies, anti-PD-L1 antibodies, anti-CTLA4 antibodies, variants thereof, antigen-binding domains thereof and combinations thereof.

In a particular embodiment, the oxazaphosphorine derivative is of formula (IIa) or (IIb) as described above wherein n is 1, and the immune checkpoint inhibitor is selected from the group consisting of anti-PDL1 antibodies, anti-PD1 antibodies, variants thereof, antigen-binding domains thereof and combinations thereof.

In a particular embodiment, the therapeutic combination of the invention is characterized in that
the oxazaphosphorine derivative is selected from geranyloxy-CPA, methylated geranyloxy-CPA, geranyloxy-IFO, methylated geranyloxy-IFO, pharmaceutically acceptable salts or solvates thereof; preferably geranyloxy-IFO, methylated geranyloxy-IFO, pharmaceutically acceptable salts or solvates thereof; and/or
the immune checkpoint inhibitor is selected from the group consisting of pembrolizumab, nivolumab, cemiplimab, Camrelizumab, sintilimab, spartalizumab, Tislelizumab, pidilizumab, JS001, avelumab, atezolizumab, durvalumab, KN305, Ipilimumab, tremelimumab, variants thereof, antigen-binding fragments thereof and combinations thereof, preferably from the group consisting of pembrolizumab, nivolumab, cemiplimab, Camrelizumab, sintilimab, spartalizumab, Tislelizumab, pidilizumab, JS001, avelumab, atezolizumab, durvalumab, and KN305, more preferably pembrolizumab, nivolumab, variants thereof, and antigen-binding fragments thereof.

In a particular embodiment, the therapeutic combination of the invention is characterized in that
the oxazaphosphorine derivative is of formula (IIa) or (IIb) as described above with R is CH₃; and
the immune checkpoint inhibitor is selected from the group consisting of PD1 inhibitors, PD-L1 inhibitors, CTLA-4 inhibitors, TIGIT inhibitors, LAG-3 inhibitors, TIM-3 inhibitors and OX-40 agonists, more preferably from anti-PD1 antibodies, anti-PD-L1 antibodies, anti-TIGIT antibodies and anti-CTLA-4 antibodies, and even more preferably from anti-PD1 antibodies and anti-PD-L1 antibodies.

In another embodiment, the therapeutic combination of the invention is characterized in that
the oxazaphosphorine derivative is of formula (IIa) or (IIb) as described above with R is H; and
the immune checkpoint inhibitor is selected from the group consisting of PD1 inhibitors, PD-L1 inhibitors, CTLA-4 inhibitors, TIGIT inhibitors, LAG-3 inhibitors, TIM-3 inhibitors and OX-40 agonists, more preferably from anti-PD1 antibodies, anti-PD-L1 antibodies, anti-TIGIT antibodies and anti-CTLA-4 antibodies, and even more preferably from anti-PD1 antibodies and anti-PD-L1 antibodies.

In another embodiment, the therapeutic combination of the invention is characterized in that
the oxazaphosphorine derivative is of formula (IIa) or (IIb) as described above with R is CH₃ and n is 1; and
the immune checkpoint inhibitor is selected from the group consisting of PD1 inhibitors, PD-L1 inhibitors, CTLA-4 inhibitors, TIGIT inhibitors, LAG-3 inhibitors, TIM-3 inhibitors and OX-40 agonists, more preferably from anti-PD1 antibodies, anti-PD-L1 antibodies, anti-TIGIT antibodies and anti-CTLA-4 antibodies, and even more preferably from anti-PD1 antibodies and anti-PD-L1 antibodies.

In another embodiment, the therapeutic combination of the invention is characterized in that
the oxazaphosphorine derivative is of formula (IIa) or (IIb) as described above with R is H and n is 1; and
the immune checkpoint inhibitor is selected from the group consisting of PD1 inhibitors, PD-L1 inhibitors, CTLA-4 inhibitors, TIGIT inhibitors, LAG-3 inhibitors, TIM-3 inhibitors and OX-40 agonists, more preferably from anti-PD1 antibodies, anti-PD-L1 antibodies, anti-TIGIT antibodies and anti-CTLA-4 antibodies, and even more preferably from anti-PD1 antibodies and anti-PD-L1 antibodies.

In an additional embodiment, the therapeutic combination of the invention is characterized in that
the oxazaphosphorine derivative is selected from geranyloxy-IFO, methylated geranyloxy-IFO, pharmaceutically acceptable salts or solvates thereof; and
the immune checkpoint inhibitor is an anti-PD1 or an anti-PD-L1 antibody.

Implementation of the Combination Therapy of the Invention

The oxazaphosphorine derivative and the immune checkpoint modulator may be administered by any conventional route. The route of administration of the oxazaphosphorine derivative and that of the immune checkpoint modulator may be the same or may be different. The administration route may be topical, parenteral, or enteral. Indeed, the therapeutic agents of the invention may be administered by any conventional route including, but not limited to, oral, buccal, sublingual, rectal, intravenous, intra-muscular, subcutaneous, intra-osseous, dermal, transdermal, mucosal, transmucosal, intra-articular, intra-cardiac, intra-cerebral, intra-peritoneal, intratumoral, intranasal, pulmonary, intraocular, vaginal, or transdermal route.

The administration route of the oxazaphosphorine derivative and that of the immune checkpoint modulator may vary depending on the properties of said therapeutic agent (in particular its bioavailability), the cancer to treat, and the organ or tissue of the patient afflicted by the cancer. In some embodiments, the oxazaphosphorine derivative is administered by oral route or intravenous route, e.g. by bolus injection or by continuous infusion. The immune checkpoint modulator, especially antibodies such as OX40 antibodies, anti-PD1 antibodies, anti-PD-L1 antibodies, anti-TIGIT antibodies, anti-LAG-3 antibodies, anti-TIM-3 antibodies, and anti-CTLA4 antibodies, may be administered by parenteral route, preferably by intravenous route e.g. by injection or infusion.

The dose regimen of the therapeutic combination of the invention may be determined and adapted by the one skilled in the art in view of the specific features of the subject, namely his/her age, gender, ethnic group, weight, health and physical condition, medical history, type of cancer and its stage, previous possible anticancer treatments, the presence of specific biomarker (such as PD-L1 expression level in tumour cells), and other relevant features.

The immune checkpoint modulator and the oxazaphosphorine derivative are administered at an effective therapeutic dose to the subject. As used herein, “a therapeutically effective amount or dose” refers to an amount of a therapeutic agent which prevents, removes, slows down a disease of interest or reduces or delays one or several symptoms or disorders caused by or associated with said disease in the subject. The dose may be administered in one, two or more boluses, tablets or injections. In certain embodiments, the therapeutic agent(s) are administered by a single injection or by infusion over an extended period of time (e.g. from 1 to 24 hours)

Typically, the amount of the oxazaphosphorine derivative to be administrated to a patient may range from about 0.01 mg/kg to 500 mg/kg of body weight, preferably from 0.1 mg/kg to 300 mg/kg of body weight, for instance from 25 to 300 mg/kg.

In some embodiments, the oxazaphosphorine derivative is administered at a therapeutic dose enabling to achieve immunomodulatory effects in the patient such as Th1 polarization and thus a T cell-dependent antitumor effects, without inducing a significant lymphopenia. This therapeutic dose varies depending on the patient, especially depending on her/his age, health condition and immune status. The daily therapeutic dose of oxazaphosphorine derivative providing immunomodulatory effects may be assessed by administering escalating low doses of the derivative to the patient (or a group of representative patients suffering from the same type of cancer) and monitoring the effects of the administered doses on lymphocyte population (e.g. B cells, natural killer T cells, T cells and Tregs) and the polarization of the immune response. For example, the skilled artisan may adapt the protocol described in Ghiringhelli, Cancer Immunol Immunother, 2007, 56:461-648).

The oxazaphosphorine derivative may be administered as single daily dose, e.g. by injection, over several consecutive days, for example during 2 to 10 consecutive days, preferably from 2 to 6 consecutive days. Alternatively, the oxazaphosphorine derivative may be infused over several consecutive hours, for instance during 6 h to 48 h, e.g. during 12 to 24 h. Said treatment may be repeated every one, two or three weeks or every one, two or three months. For instance, the treatment with the oxazaphosphorine derivative may be repeated every 3 weeks or every 6 weeks.

The treatment may be repeated one or several times per year.

The amount of the immune checkpoint modulator to be administrated to a patient may range from about 0.001 mg/kg to 100 mg/kg of body weight. When an immune checkpoint modulator is an antibody, the cumulated dose administered to a patient over a single cycle of treatment may range from 10 mg to 1 g, for instance from 100 mg to 600 mg. Said therapeutic agent may be administered as single daily dose, e.g. by injection or infusion, or in multiple doses over an extended period, e.g. over 1 to 12 weeks, for instance 1 to 8 weeks, also e.g. by injection or infusion. The frequency of administration may be one administration every month, every two weeks, every week, every two days or once daily. Said treatment may be repeated one or several times per year.

As mentioned above, the administration of the immune checkpoint modulator and that of the oxazaphosphorine derivative can be performed simultaneously, separately, consecutively, concomitantly, or successively. A combination therapy does not necessary require the therapeutic agents to be administered at the same time, in a single pharmaceutical composition, in the same pharmaceutical dosage form and/or by the same administration route. The therapeutic agents of the invention can be administered in any order to the patient.

In some embodiments, the administration of the immune checkpoint modulator and that of the oxazaphosphorine derivative are performed within the same month, the same week and even the same day to the patient. In some particular embodiments, the administration of the oxazaphosphorine derivative is performed in the month, the fortnight or the week be fore or after the administration of immune checkpoint modulator. As a further example, the oxazaphosphorine derivative may be administered 1, 2, 3, 4, 5, 6, 7, 8, 9, 10, 11, 12, 13, 14 or 15 day(s) before or after the administration the immune checkpoint modulator to the patient. In some embodiments, the administration of the therapeutic agents are performed so as to result in exposure of the subject to both therapeutic agents during a period of time based on the pharmacokinetics and clearance of the drugs.

In some embodiments, the administration of therapeutic agents are repeated every 3 weeks or every 6 weeks. The administration of the oxazaphosphorine derivatives and that of the immune checkpoint modulator may be performed the same day or be 1, 2, 3, 5, 6, 7, 8, 9, 10, 11, 12, 13, 14, 15 days apart.

In some other or additional embodiments, several (e.g. 1, 2, 3, 4 or 5) administrations of the immune checkpoint modulator are performed between two consecutive administrations of the oxazaphosphorine derivative. In some additional embodiments, several (e.g. 1, 2, 3, 4 or 5) administrations of the oxazaphosphorine derivative are performed between two consecutive administrations of the immune checkpoint modulator.

The oxazaphosphorine derivative and the immune checkpoint modulator can be administered within the same pharmaceutical composition or in different pharmaceutical compositions to the patient.

The therapeutic agents of the invention, namely the oxazaphosphorine derivative and/or the immune checkpoint modulator may be formulated in any appropriate pharmaceutical composition according to standard methods such as those described in Remington: The Science and Practice of Pharmacy (Lippincott Williams & Wilkins; Twenty first Edition, 2005). Pharmaceutically acceptable excipients that may be used are, in particular, described in the Handbook of Pharmaceuticals Excipients, American Pharmaceutical Association (Pharmaceutical Press; 6th revised edition, 2009). Typically, the therapeutic agent(s) is mixed with one or several excipients so as to obtain the desired pharmaceutical form.

Examples of appropriate excipients include, but are not limited to, solvents such as water or water/ethanol mixtures, fillers, carriers, diluents, binders, anti-caking agents, plasticizers, disintegrants, lubricants, flavors, buffering agents, stabilizers, colorants, dyes, anti-oxidants, anti-adherents, softeners, preservatives, surfactants, wax, emulsifiers, wetting agents, and glidants. Examples of diluents include, without being limited to, microcrystalline cellulose, starch, modified starch, dibasic calcium phosphate dihydrate, calcium sulfate trihydrate, calcium sulfate dihydrate, calcium carbonate, mono- or disaccharides such as lactose, dextrose, sucrose, mannitol, galactose and sorbitol, xylitol and combinations thereof. Examples of binders include, without being limited to, starches, e.g., potato starch, wheat starch, corn starch; gums, such as gum tragacanth, acacia gum and gelatin; hydroxypropyl cellulose, hydroxyethyl cellulose, hydroxypropyl methyl cellulose; polyvinyl pyrrolidone, copovidone, polyethylene glycol and combinations thereof. Examples of lubricants include, without being limited to, fatty acids and derivatives thereof such as calcium stearate, glyceryl monostearate, glyceryle palmitostearate magnesium stearate, zinc stearate, or stearic acid, or polyalkyleneglycols such as PEG. The glidant may be selected among colloidal silica, dioxide silicon, talc and the like. Examples of disintegrants encompass, without being limited to, crospovidone, croscarmellose salts such as sodium croscarmellose, starches and derivatives thereof. Examples of surfactants encompass, without being limited to, simethicone, triethanolamine, les polysorbate and derivatives thereof such as Tween® 20 or Tween® 40, poloxamers, fatty alcohol such as laurylic alcohol, cetylic alcohol, phospholipids and alkylsulfate such as sodium dodecylsulfate (SDS). Example of stabilizers, in particular useful for lyophilization encompass stabilizers typically encompass sugars such as mannitol, sucrose, dextrose and trehalose, amino-acids, hydroxypropyl-β-cyclodextrin and serum albumin. Examples of emulsifiers, encompass for example, ethyl alcohol, isopropyl alcohol, ethyl carbonate, ethyl acetate, benzyl alcohol, benzyl benzoate, propyleneglycol, 1,3-butyleneglycol, dimethylformamide, oils, polyethyleneglycol and fatty acid esters of sorbitan or mixtures of these substances. Preservatives encompass, without being limited to, benzalkonium chloride, benzoic acid, sorbic acid and salts thereof. Antioxidants encompass ascorbic acid, ascorbyl palmitate, tocopherol and combinations thereof. Examples of buffering agents encompass phosphoric acid, Tris(hydroxymethyl)aminomethane hydrochloride (TRIS·HCl), 4-Morpholinepropanesulfonic acid (MOPS), 4-(2-Hydroxyethyl)piperazine-1-ethanesulfonic acid (HEPES), PIPES, 2,2-Bis(hydroxymethyl)-2,2′,2″-nitrilotriethanol (BIS-TRIS), TRIS-glycine, Bicine, Tricine, TAPS, TAPSO, MES, citrate, borate, citrate/phosphate, bicarbonate, glutaric acid, succinic acid, salts thereof and combinations thereof.

It goes without saying that the excipient(s) to be combined with the therapeutic agent of interest may be selected taking into account (i) the physico-chemical properties including the stability of said therapeutic agent, (ii) the pharmacokinetic profile and/or the release profile sought for said therapeutic agent, (iii) the dosage form and (iv) the route of administration.

The pharmaceutical composition may be of any type. For instance the pharmaceutical composition may be a solid oral dosage form, a liquid dosage form, a suspension, for instance for intravenous route, a dosage form for topical application such as cream, ointment, gel and the like, a patch, such as a transdermal patch, a muco-adhesive patch or tablet, in particular adhesive plaster or bandage, a suppository, an aerosol for intranasal or pulmonary administration . . . . In some particular embodiments, the pharmaceutical composition may be a lyophilizate or a freeze-dried powder. The powder may comprise a therapeutic agent of the invention (i.e. the immune checkpoint modulator and/or the oxazaphosphorine derivative) combined with one or several excipients selected from buffer agent, freeze-drying stabilizer, anti-oxidant, surfactant and combinations thereof. Said powder may be dissolved or suspended in an appropriate vehicle, e.g. in water, just before being administered to the patient, for instance by intravenous route (e.g. by bolus injection or infusion) or by oral route.

In an additional aspect, the therapeutic combination of the invention (e.g. the oxzaphosphorine derivative and the immune checkpoint modulator) may be administered to the subject in combination with an additional therapeutic agent. The additional therapeutic agent may be an anticancer agent. Non-limiting examples include in particular interferons, cisplatin, bleomycin, fluorouracil, methotrexate, vincristine, actinomycin, vinorelbine, taxanes such as paclitaxel and docetaxel, or an anthracycline. Furthermore, an active ingredient for neutralizing the potential toxicity of acrolein can be administered, in particular sodium mercaptoethanesulfonate. The additional therapeutic agent may be administered to the patient by the same route or by distinct routes, simultaneously, separately or successively with respect to the immune checkpoint modulator and/or the oxazaphosphorine derivative.

The therapeutic combination of the invention may be also used in a patient cotreated with radiotherapy.

In a further aspect, the invention relates to a pharmaceutical composition, preferably for use in the treatment or the prevention of a cancer, which comprises an immune checkpoint modulator and a oxazaphosphorine derivative as active ingredients. Its goes without saying that the immune checkpoint modulator and the oxazaphosphorine derivative are as described above.

In some embodiments, the pharmaceutical composition of the invention comprises:
from 0.01% to 45% by weight of an oxazaphosphorine derivative,
from 0.01% to 45% by weight of an immune checkpoint modulator, and
from 50% to 99.98% by weight of one or several pharmaceutical excipients.

The one or several pharmaceutical excipients can be of any type, and may be selected from the group consisting of carriers, diluents, binders, surfactants, stabilizers, antioxidants, preservatives, disintegrants and combinations thereof.

The pharmaceutical compositions may be of any type as described above. Pharmaceutical compositions suitable for parenteral injection, e.g. by intravenous route, may be preferred.

In an additional aspect, the invention refers to a pharmaceutical kit preferably for use in the treatment or the prevention of a cancer, said kit comprising at least two components:
a first component comprising at least one oxazaphosphorine derivative as described above, and
a second component comprising at least one immune checkpoint modulator as described above.

Preferred oxazaphosphorine derivatives and immune checkpoint modulators are those described hereabove. For instance the at least one oxazaphosphorine derivative may be selected from compounds of formula (Ia), (IIa) and (IIb) as described above and/or the immune checkpoint modulator may be selected from PD1 inhibitors, PD-L1 inhibitors, CTLA4 inhibitors and combinations thereof, preferably anti-PD1 antibodies, anti-PD-L1 antibodies, anti-CTLA4 antibodies and combinations thereof.

In a particular embodiment, the pharmaceutical kit comprises at least three components:
a first component comprising at least one oxazaphosphorine derivative selected from compounds of formula (Ia), (IIa), and (IIb) as described above,
a second component comprising an anti-PD1 or an anti-PD-L1 antibody, and
an optional third component comprising an anti-CTLA4 antibody.

Typically, said components are in the form of pharmaceutical compositions as described above.

The pharmaceutical compositions may be packed in sterile containers. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The pharmaceutical kit may comprise further elements such as buffers, means for the administration of the components (e.g. means for administration by bolus injection or infusion such as syringe, needle, catheter and the like), and label including instructions for use.

Other aspects of the present invention are illustrated in the following examples, which are only illustrative in nature, but do not limit the scope of this application.

### EXAMPLES

Materials and Methods

Chemical Agents and Reagents

Cyclophosphamide (CPA) (Endoxan®; Baxter) and ifosfamide (IFO) (Holoxan®; Baxter) were provided by Gustave Roussy Cancer Campus Grand Paris. Geranyloxy-IFO (G-IFO) was synthesized with 99% purity as previously described in Sharbek (Journal of Medecinal Chemistry, 2015, 58(2):705-17). For in vivo studies, CPA and IFO were dissolved in NaCl 0.9% or DMSO/Tween 80/NaCl 0.9% (5/5/90, v/v/v). G-IFO was dissolved in DMSO/Tween 80/NaCl 0.9% (5/5/90, v/v/v). Monoclonal anti-CD4 (GK1.5), anti-CD8α (53-6.72), anti-PD1 (RMP1-14) and their isotype control rIgG2a (2A3) for in vivo experiments were purchased from BioXCell (West Lebanon, NH, USA) and dissolved in Phosphate-buffered saline (PBS). Monoclonal antibodies (mAbs) used for flow cytometry and immunohistochemistry analysis are described in Table 51.

Mice and Tumor Cell Line

7-8 weeks-old female C57BL/6 mice (mean body weight, 20 g) were purchased from Harlan Laboratories (Gannat, France). Animals were used in pathogen-free conditions. MCA205 fibrosarcoma tumor cell line (syngenic from C57Bl/6 mice) was kindly provided by Dr Yamazaki Takahiro (INSERM U1015, Gustave Roussy, Villejuif, France). They were harvested at 37° C. under 5% CO₂ in Gibco™ RPMI 1640 medium (Paisley, UK) supplemented with 10% Fetal Bovine Serum (Paisley, UK) and 2 mM L-glutamine (Invitrogen, USA). All animal experiments were carried out in compliance with French and European laws and regulations and by the CEEA26 Ethic committee and the ministry of national education, higher education and research and carried out under conditions established by the European Community (Directive 2010/63/2015-038).

Tumor Model and Tumor Inoculation in Mice

8·10⁵ tumor cells were inoculated subcutaneously (s.c.) in the right flank of C57Bl/6 mice on DO. Mice received a single intraperitoneal (i.p.) injection of CPA at 100 mg/kg, IFO at 100, 150, 200 or 300 mg/kg, vehicle or G-IFO at equimolar dose of IFO 50, 100 or 150 mg/kg when the tumor volume reach a size between 50 and 500 mm3 (V (mm3)=width2 (mm2)×length (mm)/2). For T cell depletion, mice received 200 μg/mouse i.p. injections of anti-CD8α (clone 53-6.72) and/or anti-CD4 (clone GK1.5) or their isotype control Rat IgG2a (clone 2A3) on Days(D)-3, D0, D+3 then once a week, and IFO or control on D7.

For combination between chemotherapy (IFO) and anti-PD1 mAbs, mice received IFO or vehicle on D7 then 250 μg/mouse i.p. injections of anti-PD1 mAbs (clone RMP1-14) or its isotype control Rat IgG2a (clone 2A3) on D9, D12 and D15. Regarding study for combination between chemotherapy (G-IFO) and anti-PD1 mAbs, mice received G-IFO or vehicle on D9 then 200 μg/mouse i.p. injections of anti-PD1 mAbs (clone RMP1-14) or its isotype control Rat IgG2a (clone 2A3) on D12, D15 and D19. Tumor volume was followed-up three times a week by measuring the length and width using a caliper. In order to normalize tumor measurements for each day VT_{Dx} to VT_{Di} (VT_{Dx}/VT_{Di}) ratio was calculated; VTDi corresponds to the tumor volume the day of treatment initiation and VT_{Dx} corresponds to the tumor volume on each measurement day for each mouse.

Flow Cytometry Analysis

Female C57BL/6 mice of 7 to 8 weeks were randomly assigned to the different treatment groups. Six groups of mice were evaluated including an untreated control group receiving the vehicle and four to five treated groups with IFO at the dose of 100, 150, 200 and 300 mg/kg and CPA at 100 mg/kg. Both drugs were dissolved in a solution of NaCl 0.9%. Adding groups treated with G-IFO at the equimolar dose of IFO 50, 100 and 150 mg/kg, vehicle, CPA, IFO and G-IFO were dissolved in a solution of DMSO/Tween 80/NaCl 0.9% (5/5/90, v/v/v). The administrations were performed by a single i.p. injection with a volume of 20 mL/kg or 10 mL/kg when adding G-IFO groups. Seven days post treatment, the mice were sacrificed, spleens and tumors were collected. After lysis of red blood cells with ammonium chloride splenic viable cells were quantified with Trypan Blue with Vi-CELL XR (Beckman Coulter).

Briefly, tumor dissociation was done with GentleMACS™ Dissociator after adding ADNase (260913, Millipore) and ligase (5401127001, Sigma) to weighed and cut tumors. Tumor cells were incubated for 40 min at 37° C. under stirring and then quantified with Trypan Blue with Vi-CELL XR (Beckman Coulter). Before staining, Fcγ-receptors were blocked for 15 min at 4° C. using anti-CD16/32 functional grade purified antibodies (eBioscience, Paris, France). Cells were incubated for 30 min at 4° C. with antibodies for cell surface staining. For FoxP3 staining, cells were fixed and permeabilized after cell surface staining according to the FoxP3 kit protocol (eBioscience, Paris, France). Samples were acquired on 10-colors Gallios cytometer (Beckman Coulter, Villepinte, France). Analyses were performed using Kaluza software 1.3 (Beckman Coulter). Two different panels were used to identify immune cells. First leucocytes were identified by the use of FITC-conjugated anti-mouse CD45. T and B lymphocytes were identified using APC-Cy7-conjugated anti-mouse CD3 and BV421-conjugated anti-mouse CD19 respectively. CD4⁺ and CD8⁺ T cells were separated using PE-Cy7-conjugated anti-mouse CD4 and APC-R700-conjugated anti-mouse CD8a staining among CD3 positive cells, respectively. Treg cells were stained using APC-conjugated anti-mouse FoxP3 staining among CD3⁺CD4⁺ T cells (Table 1).

TABLE 1
Antibodies used for flow cytometry experiments

| Antigen | Species | Clone | Fluorochrome | Supplier |
|---|---|---|---|---|
| CD45 | mouse | 30-F11 | FITC | BD biosciences |
| CD3e | mouse | 145-2C11 | APC-Cy7 | BD biosciences |
| CD4 | mouse | RM4-5 | PC7 | BD biosciences |
| CD8a | mouse | 53-6.7 | APC-R700 | BD biosciences |
| FoxP3 | mouse | FJK-16s | APC | eBioscience |
| CD25 | mouse | PC61 | PE | BD biosciences |
| CD19 | mouse | 6D5 | BV421 | BioLegend |

Cytokine Assay

From spleen cell suspension, a total of 2·10⁵ cells per well were incubated in 96 well Nunc MaxiSorp® plates (eBioscience) precoated with anti-CD3ε mAbs (clone 145-2C11, 10 μg/mL); eBioscience) and/or anti-CD28 mAbs (clone 37.57, 2 μg/mL; BD Pharmingen). The supernatants were assayed after 48 hours of incubation at 37° C. under 5% CO2 using Cytokine concentrations were quantified in the supernatant using a Bio-Plex™ Mouse Cytokine Standard 23-Plex, Group I Assay (bio rad, M60009RDPD). The panel was comprised of the following cytokines and chemokines: eotaxin, Granulocyte-Colony Stimulating Factor (G-CSF), Granulocyte-Macrophage-Colony Stimulating Factor (GM-CSF), interferon gamma (IFNγ), interleukin (IL)-1α (IL-1α), IL-1β, IL-2, IL-3, IL-4, IL-5, IL-6, IL-9, IL-10, IL-12p40, IL-12p70, IL-13, IL-17A, Keratinocyte Chemoattractant (KC), Macrophage Chemotactic Protein-1 (MCP-1), Macrophage Inflammatory Protein (MIP)-1alpha (MIP-1α), MIP-1β, Regulated on Activation Normal T cell Expressed and Secreted (RANTES) and Tumor Necrosis Factor-alpha (TNF-α). Results were analyzed using Bio-Plex Manager Software V 6.1 (Bio-Rad Laboratories, Hercules, CA, USA). Selection of cytokines and chemokines that were significantly regulated after IFO treatment in mice allows us to reduce the monitoring to IFNγ, IL-17A and IL-6. These three cytokines were then quantified using mouse IL-17A ELISA Ready-SET-Go® (eBiosciences), Mouse IFNy ELISA Set (BD Biosciences) and Mouse IL-6 ELISA Set (BD Biosciences).

Statistical Analysis

Data were analyzed with Microsoft Excel® (Microsoft Co., Redmont, WA, USA), Prism™ 5.0 and 8.0 software (GraphPad San Diego, CA, USA). All results are expressed as mean±standard error of mean or median with interquartile. Statistically significant differences were analyzed using the non-parametric Mann-Whitney test or the non-parametric Kruskall-Wallis test to compare more than two independent groups, two-way ANOVA test to compare groups with two independent variables coupled to Geisser-Greenhouse for correction of violation of sphericity in repeated measures. No adjustment for multiple comparisons was made with small populations (n≤6) because of the exploratory component of the analyses. A p-value smaller than 0.05 was considered to be statistically significant. Significant p values were annotated as follows *p<0.05, **p<0.01, ***p<0.001, ****p<0.0001.

Results

Immunomodulatory Effects of IFO and Geranyloxy-IFO

The effect of escalating doses of IFO on antitumor response and immune response was explored. CPA (cyclophosphamide), was used at 100 mg/kg since previous study demonstrated its immune-mediated antitumor response at this dose.

Antitumor activity of escalating single i.p. injection of IFO (100, 150, 200 and 300 mg/kg) or of CPA (100 mg/kg) in the immunocompetent MCA205-bearing C57Bl/6 mice was assessed. Significant reduction of the tumor growth was observed for CPA at 100 mg/kg; for IFO, a delay of tumor growth from low doses (100 and 150 mg/kg) to higher doses (200 and 300 mg/kg) were also observed.

In naïve mice, IFNγ, IL-17A and IL-6 were significantly increased after treatments. As expected the vehicle displayed weak cytokine secretions. In mice treated with CPA at 100 mg/kg, TCR-driven IFNγ, IL-17A and IL-6 were significantly increased as previously published. Regarding the IFO groups (100, 150 and 200 mg/kg), a significant increase of TCR-driven IFNγ, IL-17A and IL-6 was also observed after CD3ε+CD28 stimulation.

As for naive mice, the Inventors examined T cell polarization after TCR engagement in MCA205 tumor-bearing mice. The known cytotoxic dose of IFO i.e. 300 mg/kg was added to the experiment in tumor-bearing mice; IFO 200 and 300 mg/kg failed to induce IL-17A and IFNγ TCR-driven cytokines, only TCR-driven IL-6 remained highly secreted. These results are reminiscent of the T cell counts diminution as well as decrease of T cell proportion observed at higher doses. For IFO at 100 and 150 mg/kg where no T cell counts decrease was observed in tumor-bearing mice, significant secretion of TCR-driven IL-17A, IFNγ and IL-6 were detected after CD3ε and after CD28 co-stimulation. Unexpectedly IFO 150 mg/kg induced more TCR-driven IL-17 and IL-6 than CPA 100 mg/kg.

Complementary study was performed in order to confirm the T cells involvement in antitumor activity for IFO at low dose. MCA205-bearing mice were depleted in both CD4+ and CD8+ T cells, and treated with a single i.p. injection of IFO at 150 mg/kg. Significant reduction of the tumor growth was observed for non-depleted mice. For CD4+ T cells and CD8+ T cells depleted mice, the Inventors observed a decrease of the antitumor effect. Finally, antitumor efficacy of IFO 150 mg/kg was completely abolished in mice depleted with both CD4+ and CD8+ T cells. Altogether, these data indicated that at low dose of IFO (150 mg/kg), T cells are mandatory to observe antitumor immune-mediated effect.

These results on immune-mediated antitumor response of IFO led the Inventors to study immunomodulatory properties on the less toxic oxazaphosphorine derivative, G-IFO.

In the herein described experiments, the dose of G-IFO is defined as the equivalent molar dose of IFO (eq. X mg/kg). For instance, 40 mg/kg of G-IFO is equivalent to 25 mg/kg of IFO as the molar masses are 419 g/mol for G-IFO and 261 g/mol for IFO.

The Inventors assessed the antitumor activity of single i.p. injection of G-IFO at eq. 100 mg/kg in the immune competent MCA205-bearing C₅₇Bl/6. The dose of G-IFO at eq. 100 mg/kg did not show cytotoxicity on T cell populations in spleens (FIG. 2A) and in tumor (FIG. 2B) compared to higher dose of G-IFO (eq. 150 mg/kg). As shown in FIG. 2D, significant delay of the tumor growth was observed for the three molecules with a lower tumor growth delay for G-IFO compared to CPA 100 mg/kg. These data suggest that G-IFO is able to delay tumor growth even at a single low dose.

The Inventors also investigated the TCR-driven cytokine release in MCA205-bearing mice for the escalating doses of G-IFO. As shown in FIG. 2B, G-IFO eq. 150 mg/kg induced high levels of IL-6 but poor secretion of IFNγ whereas G-IFO eq. 100 mg/kg favored IFNγ secretion i.e. Th1 polarization. No significant IL-17 secretion could be observed in these experiments with G-IFO.

Altogether, these results showed that G-IFO at eq. 150 mg/kg induced T cell depletion probably limiting Th1 accumulation while G-IFO eq. 100 mg/kg showed an antitumoral activity, did not affect the numbers of T cells and demonstrated an increase of IFNγ and IL-6 secretions. Therefore, G-IFO at eq. 100 mg/kg was selected as the immunomodulatory dose.

Synergy Between Anti-PD1 Antibody, Oxazaphosphorines and Pre-Activated Oxazaphosphorines (X-Oxaza; i.e. G-IFO)

As shown in FIG. 3 (A-B), anti-PD1 mAbs were not able to reduce tumor growth in the MCA205 tumor model when administered alone.

No improvement of the antitumor efficacy was observed with IFO at high cytotoxic dose (300 mg/kg) or at the immunomodulatory dose (150 mg/kg) when IFO is associated to anti-PD1 mAbs (FIG. 4). In contrast, the oxazaphosphorine derivative of the invention, geranyloxy-IFO at low dose (G-IFO eq. 100 mg/kg) was shown to highly enhance the antitumor efficacy in combination with anti-PD1 mAbs (FIG. 3). Total tumor regressions were observed in 17% of the mice 3).

Finally, the time to reach five-fold the initial volume was highly delayed with G-IFO eq. 100 mg/kg+anti-PD1 mAbs compared to G-IFO eq. 100 mg/kg alone and anti-PD1 mAb alone showing a synergic effect of the combination of G-IFO with anti-PD1 antibodies on tumor growth (FIG. 3B).

### CONCLUSION

MCA205 was poorly responding to anti-PD-1 mAbs as a standalone treatment. As shown on FIG. 4, adding a single injection of G-IFO at eq. 100 mg/kg to anti-PD1 mAbs treatment improved the antitumor efficacy. Interestingly, a potent synergy was observed when anti-PD1 mAbs were associated with G-IFO eq. 100 mg/kg. Thus G-IFO at low dose seemed pertinent to leverage anti-PD-1 mAbs activity. Such effects were not observed with IFO at both high and low doses.

The Inventors further scrutinized immune modifications following i.p. injection of G-IFO in mice. B cells population seemed very affected by G-IFO even at low dose of G-IFO (eq. 100 mg/kg) (FIG. 4) underlining high sensitivity of B cells to the direct killing by these cytotoxic agents as previously reported. Such B cells decrease could be an advantage when using an oxazaphosphorine derivative with an immune checkpoint inhibitor. Indeed, immune checkpoint inhibitors such as anti-PD-1, anti-PD-L1 and anti-CTLA-4 antibodies have frequent immune-related adverse events (irAEs). Some of these irAEs are the consequence of auto-antibodies induction and/or increase. Nowadays the corticoids administration is the main treatment for serious irAEs with interruption of immunotherapy in most of the case. Thus, combining immune checkpoint modulators with oxazaphosphorine derivatives of the invention could lead to less frequent reactivation of auto-reactive B cells and thus less autoimmune-related adverse events.

1-18. (canceled)

19. A method of treating cancer comprising the administration of an oxazaphosphorine derivative of formula (I): 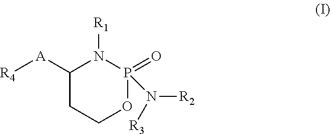
wherein:
A is O, O—O, S, NH, NR₅ with R₅ is an alkyl group or a linker group having a molecular weight up to 500 g·mol⁻¹,
R₁, R₂ and R₃ are independently selected from the group consisting of —H, —CH(CH₃)—CH₂—X and —(CH₂)₂—X, wherein X is a halogen atom,
R₄ is H or a saturated or unsaturated chain of 2 to 30 carbon atoms optionally interrupted by one or several heteroatoms selected from S, O and NH, and optionally substituted by one or several substituents independently selected from the group consisting of halogen, CN, CF₃, OH, C₁-C₆ alkyl, C₁-C₆ hydroxyalkyl, C₁-C₆ alkyloxy, C₁-C₆ aminoalkyl, C₁-C₆ halogenoalkyl, —C₂-C₆ alkoxy alkyl, —C(O)OR, —OC(O)R, —OC(O)OR, —C(O)R,
—NHC(O)—NH—R, —NH—C(O)—R, —C(O)—NH—R, —NRR′, —C(O)NRR′, —NC(O)R, —NRC(O)R′, and —SR, wherein R and R′ are independently selected from H and C₁-C₆ alkyl,
and pharmaceutically acceptable salt or solvate thereof,
in combination with an immune checkpoint modulator to a subject in need of treatment.

20. The method according to claim 19, wherein the oxazaphosphorine derivative is of formula (Ia): 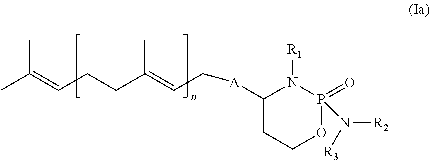
wherein:
n is an integer from 0 to 3,
A, R₁, R₂ and R₃ are:
A is O, O—O, S, NH, NR₅ with R₅ is an alkyl group or a linker group having a molecular weight up to 500 g·mol⁻¹,
R₁, R₂ and R₃ are independently selected from the group consisting of —H, —CH(CH₃)—CH₂—X and —(CH₂)₂—X, wherein X is a halogen atom,
and pharmaceutically acceptable salts and solvates thereof.

21. The method according to claim 20, wherein:
n is 1 or 2,
A is selected from the group of O, O—O, S, and —NH—, or comprises, or consists of, a spacer moiety selected from the group consisting of:
natural or non-natural amino acids, dipeptides, and derivatives thereof,
polyether groups,
hydrazone linkers,
—O—(C═S)—S—, —ONR₇—, —NR₇O—, with R₇ being H or a C₁-C₆ alkyl,
Y₁—(CH₂)ₙ—Y₂, with n is an integer from 1 to 8, wherein Y₁ and Y₂ are independently selected from —O—, —S—, —OC(O)—, —C(O)O—, —OC(O)—O—, —C(O)NR₇—, NR₇C(O)—, —OC(S)S—, —SC(S)O— —NR₇—, —ONR₇—, —NR₇O—, NR₇C(S)S—, —SC(S)NR₇—, and 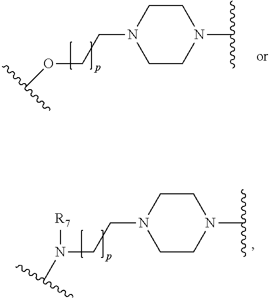
wherein R₇ is selected from the group consisting of H and C₁-C₆ alkyl and p is an integer from 0 to 8, and
one of R₁, R₂ and R₃ is H and the two other remaining groups are independently selected from —CH(CH₃)—CH₂—X and —(CH₂)₂—X.

22. The method according to claim 19, wherein A is O—O, O, S or NH, or is a moiety selected from the group consisting of:
—O—(C═S)—S—, —ONR₇—, —NR₇O—, with R₇ is H or a C₁-C₃ alkyl,
citrulline, lysine, ornithine, alanine, phenylalanine, cysteine, glycine, valine, leucine and dipeptides thereof,
Y₁—(CH₂)ₙ—Y₂, and
Y₁—(CH₂—CH₂—O)ₐ—CH₂—CH₂—Y₂,
wherein Y₁ and Y₂ are independently selected from O, NR₇, S, OC(O), C(O)O, NHCO, CONH with R₇ is H or a C₁-C₃ alkyl, n is an integer from 1 to 8, and a is an integer from 1 to 3.

23. The method according to claim 19, wherein R₁, R₂ and R₃ are independently selected from the group consisting of —H and —CH(CH₃)—CH₂—X, wherein X is a halogen atom.

24. The method according to claim 19, wherein R₁, R₂ and R₃ are independently selected from the group consisting of —H and —CH₂—CH₂—X, wherein X is a halogen atom.

25. The method according to claim 19, wherein said oxazaphosphorine derivative is selected from the group consisting of compounds of formula (IIa) and of formula (IIb): 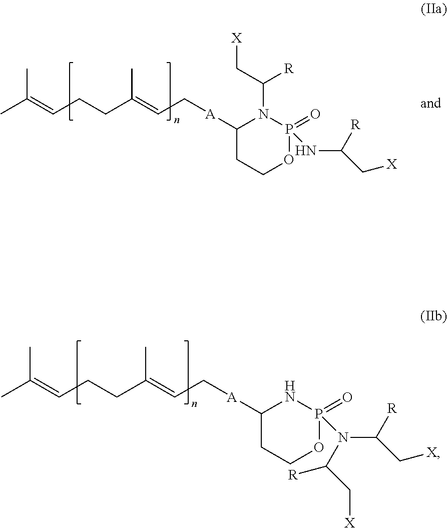
wherein:
n is 1 or 2,
R is H or CH₃,
X is Cl or Br, and
A is selected from the group consisting of O, S, —NH—, cysteamine linker, valine-citrulline linker and cysteine linker,
and pharmaceutically acceptable salts and solvates thereof.

26. The method according to claim 19, wherein the oxazaphosphorine derivative is selected from the group consisting of: 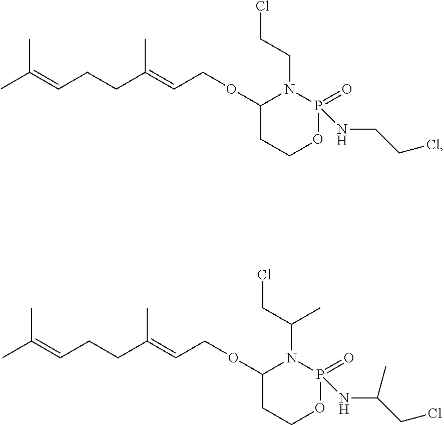 [SMILES: CC(C)=CCC/C(C)=C/COC1CCOP(=O)(NCCCl)N1CCCl ; CC(C)=CCC/C(C)=C/COC1CCOP(=O)(NC(C)CCl)N1C(C)CCl]
and pharmaceutically acceptable salts and solvates thereof.

27. The method according to claim 19, wherein the immune checkpoint modulator is an inhibitor of an immune checkpoint pathway selected from CTLA-4, PD-1, LAG-3, TIM-3, TIGIT and 2B4/CD244 immune checkpoint pathways.

28. The method according to claim 27, wherein the immune checkpoint modulator is selected from the group consisting of an anti-PD1 antibody, an anti-PD-L1 antibody, an anti-CTLA4 antibody, an anti-TIGIT antibody, and combinations thereof.

29. The method according to claim 28, wherein the immune checkpoint modulator is selected from the group consisting of pembrolizumab, nivolumab, cemiplimab, camrelizumab, sintilimab, spartalizumab, Tislelizumab, pidilizumab, JS001, avelumab, atezolizumab, durvalumab, BMS936559, MDX-1105, KN305, ipilimumab, tremelimumab, tiragulomab, vibostolimab, variants thereof, antigen-binding fragments thereof and combinations thereof.

30. The method according to claim 19, wherein the immune checkpoint modulator is an OX40 agonist.

31. The method according to claim 19, wherein the oxazaphosphosphorine derivative is geranyloxy-IFO and the immune checkpoint modulator is selected from PD1 inhibitors and PD-L1 inhibitors.

32. The method according to claim 31, wherein the immune checkpoint modulator is selected from the group consisting of pembrolizumab, nivolumab, variants thereof, antigen-binding fragments thereof and combinations thereof.

33. The method according to claim 19, wherein the oxazaphosphorine derivative and the immune checkpoint modulator are administered to the subject simultaneously, successively or separately to the subject by the same administration route or by different administration routes.

34. The method according to claim 19, wherein the cancer is selected from the group consisting of the chronic leukemias, acute lymphocytic leukemias, Hodgkin's disease, Hodgkin's and non-Hodgkin lymphomas, cancers of the lung, breast cancer, triple negative breast cancer, genitourinary cancers, cancers of prostate, bladder, testis, uterine cervix or ovaries, sarcomas, osteosarcoma, soft tissue sarcoma pediatric soft tissue sarcoma, neuroblastomas, myelomas, Merkel-cell carcinoma and melanomas.

35. A pharmaceutical composition suitable for use in the treatment or the prevention of cancer, which comprises an oxazaphosphorine derivative as defined in claim 19, and an immune checkpoint modulator.

36. A pharmaceutical kit comprising:
a first component comprising an oxazaphosphorine derivative according to claim 19, and
a second component comprising an immune checkpoint modulator.
